(19) United States
(12) Patent Application Publication
(10) Pub. No.: US 2003/0232019 A1
(43) Pub. Date: Dec. 18, 2003

(54) Inhalable formulations for sustained release

(75) Inventors: Sujit K. Basu, Cambridge, MA (US); Katharina Elbert, Cambridge, MA (US); Jeffrey Hrkach, Cambridge, MA (US); Giovanni Caponetti, Piacenza (IT)

Correspondence Address: Elmore Craig, P.C., 209 Main Street, No. Chelmsford, MA, 01863, US

(73) Assignee: Advanced Inhalation Research, Inc., Cambridge, MA

(21) Appl. No.: 10/371,398

(22) Filed: Feb. 20, 2003

Related U.S. Application Data
(60) Provisional application No. 60/427,845, filed on Nov. 20, 2002.
(60) Provisional application No. 60/359,466, filed on Feb. 22, 2002.

(51) Int. Cl. A61L 9/04; A61K 9/14

(52) U.S. Cl. 424/46; 424/489

(57) ABSTRACT

The present invention is based, in part, on the unexpected discovery that aerosol particle formulations for pulmonary delivery of a therapeutic, prophylactic or diagnostic agent comprising an asymmetric phospholipid exhibit sustained release and/or sustained action of the agent. In some embodiments, as an alternative to one or more asymmetric phospholipids or in addition to one or more asymmetric phospholipids, the instant particles comprise one or more glycerol fatty acid esters. The present invention is directed to spray dried non-polymeric particles for pulmonary delivery and sustained release of a therapeutic, prophylactic or diagnostic agent and methods for delivery of said particles to the pulmonary system, the particles comprising a therapeutic, prophylactic or diagnostic agent and an asymmetric phospholipid and/or one or more glycerol fatty acid esters. In one embodiment, the particles comprise a combination of phospholipids wherein at least one of the phospholipids is an asymmetric phospholipid. In another embodiment, the particles comprise one or more phospholipids and one or more glycerol fatty acid esters.

### RELATED APPLICATIONS

This application claims the benefit of U.S. Provisional Application No. 60/427,845, filed Nov. 20, 2002, and U.S. Provisional Application No. 60/359,466, filed Feb. 22, 2002. The entire teachings of the above applications are incorporated herein by reference.

### BACKGROUND OF THE INVENTION

Pulmonary delivery of bioactive agents, for example, therapeutic, diagnostic and prophylactic agents, provides an attractive alternative to, for example, oral, transdermal and parenteral administration. That is, typically pulmonary administration can be completed without the need for medical intervention (i.e., self-administration is available), the pain often associated with injection therapy is avoided, and the amount of enzymatic and pH mediated degradation and/or modification of the bioactive agent, frequently encountered with oral therapies, can be significantly reduced. In addition, the lungs provide a large mucosal surface for drug absorption and there is no first-pass liver effect of absorbed drugs. Further, it has been shown that high bioavailability of many molecules, for example, macromolecules, can be achieved via pulmonary delivery or inhalation. Typically, the deep lung, or alveoli, is the primary target of inhaled bioactive agents, particularly for agents requiring systemic delivery.

The release kinetics or release profile of a bioactive agent into the local and/or systemic circulation is a key consideration in most therapies, including those employing pulmonary delivery. That is, many illnesses or conditions require administration of a constant or sustained levels of a bioactive agent to provide an effective therapy. Typically, this can be accomplished through a multiple dosing regimen or by employing a system that releases the medicament in a sustained fashion.

However, delivery of bioactive agents to the pulmonary system typically results in rapid release of the agent following administration. For example, U.S. Pat. No. 5,997,848 to Patton et al. describes the rapid absorption of insulin following administration of a dry powder formulation via pulmonary delivery. The peak insulin level was reached in about 30 minutes for primates and in about 20 minutes for human subjects. Further, Heinemann, Traut and Heise teach in Diabetic Medicine 14:63-72 (1997) that the onset of action, assessed by glucose infusion rate, in healthy volunteers after inhalation was rapid with the half-maximal action reached in about 30 minutes.

As such, a need exists for formulations suitable for inhalation comprising a therapeutic, prophylactic or diagnostic agent, such as albuterol, and wherein the agent is released in a sustained fashion into systemic and/or local circulation.

### SUMMARY OF THE INVENTION

The present invention is based, in part, on the unexpected discovery that aerosol particle formulations for pulmonary delivery of a therapeutic, prophylactic or diagnostic agent comprising an asymmetric phospholipid exhibit sustained release of the agent. The present invention is directed to spray dried non-polymeric particles for pulmonary delivery and sustained release of a therapeutic, prophylactic or diagnostic agent and methods for delivery of said particles to the pulmonary system, the particles comprising a therapeutic, prophylactic or diagnostic agent and an asymmetric phospholipid. In one embodiment, the particles comprise a combination of phospholipids wherein at least one of the phospholipids is an asymmetric phospholipid. The particles of the instant invention can further comprise an amino acid. Preferably, the particles further comprise the hydrophobic amino acid leucine.

The particles of the invention are preferably aerodynamically light. In one embodiment, the particles have a tap density of less than about 0.4 g/cm³. In another embodiment, the particles have a median geometric diameter of between about 5 and 30 microns. In yet another embodiment, the particles of the invention have an aerodynamic diameter of between about 1 and about 5 microns.

In one aspect, the present invention is directed to a method for delivering a sustained release of a therapeutic, prophylactic or diagnostic via the pulmonary system, the method comprises administering to the respiratory tract of a patient in need of treatment, prophylaxis or diagnosis an effective amount of spray dried non-polymeric particles comprising a therapeutic, prophylactic or diagnostic agent; and an asymmetric phospholipid wherein the particles have a tap density of less than about 0.4 g/cm³.

Additionally, the present invention includes particles for pulmonary delivery of a therapeutic, prophylactic or diagnostic agent comprising a glycerol fatty acid ester or a combination of glycerol fatty acid esters, for example, particles for pulmonary delivery of a therapeutic, prophylactic or diagnostic agent wherein the particles comprise a therapeutic, prophylactic or diagnostic agent; a glycerol fatty acid ester or a combination of glycerol fatty acid esters; and a phospholipid or combination of phospholipids. In a preferred embodiment, the particles have a tap density of less than about 0.4. g/cm³.

The present invention also includes a method for pulmonary delivery of a therapeutic, prophylactic or diagnostic agent comprising administering an effective amount of particles comprising a glycerol fatty acid ester or a combination of glycerol fatty acid esters. For example, the invention comprises a method for delivering a sustained release of a therapeutic, prophylactic or diagnostic via the pulmonary system, the method comprising administering to the respiratory tract of a patient in need of treatment, prophylaxis or diagnosis an effective amount of particles comprising a therapeutic, prophylactic or diagnostic agent; a glycerol fatty acid ester or a combination of glycerol fatty acid esters; and a phospholipid or combination of phospholipids.

### BRIEF DESCRIPTION OF THE DRAWINGS

FIG. 1 is a plot of in vivo data showing mean enhanced pause (mean PenH) versus time, in hours, for bronchoprotection provided by a dry powder particle formulation containing an asymmetric phospholipid in a guinea pig model of methacholine induced airway hyperresponsiveness.

FIG. 2 is a plot of in vitro data showing the amount of albuterol sulfate released as a percent versus time (in hours) from three particle formulations, the particles comprising glycerol fatty acid esters (Precirol ATO5), a phospholipid, and albuterol sulfate (i.e., dry powder Formulations U, V, and W).

FIG. 3 is a plot of in vitro data showing the amount of albuterol sulfate released as a percent versus time (in minutes) from three particle formulations, the particles comprising glycerol fatty acid esters (Precirol ATO5), a phospholipid, and albuterol sulfate (i.e., dry powder Formulations NN, OO, and PP).

FIG. 4 is a plot of in vivo data showing mean enhanced pause (mean PenH) versus time, in hours, for bronchoprotection provided by a dry powder particle formulation (i.e., Formulation LL) containing glycerol fatty acid esters (Precirol ATO5), a phospholipid, and albuterol sulfate as compared to bronchoprotection provided by a liquid albuterol sulfate aerosol in a guinea pig model of methacholine induced airway hyperresponsiveness.

The foregoing and other objects, features and advantages of the invention will be apparent from the following more particular description of preferred embodiments of the invention.

### DETAILED DESCRIPTION OF THE INVENTION

The present invention is directed toward particles for pulmonary drug delivery and methods for delivering the particles to the pulmonary system. The particles and respirable compositions comprising the particles of the present invention described herein comprise a bioactive agent, such as albuterol, as a therapeutic, prophylactic or diagnostic agent and an asymmetric phospholipid. Alternatively, particles for sustained release of a therapeutic, prophylactic or diagnostic agent and respirable compositions comprising those particles can comprise a glycerol fatty acid ester or a combination of glycerol fatty acid esters. For example, particles for pulmonary delivery of a therapeutic, prophylactic or diagnostic agent wherein the particles comprise a therapeutic, prophylactic or diagnostic agent; a glycerol fatty acid ester or a combination of glycerol fatty acid esters; and a phospholipid or combination of phospholipids (e.g., the phospholipid or combination of phospholipids comprise asymmetric and/or symmetric phospholipids).

The particles and respirable compositions comprising the particles of the invention, both hereinafter referred to as “particles” or “powders,” are preferably biodegradable and biocompatible, and optionally are capable of affecting the biodegradability and/or the rate of delivery of the co-administered agents. In addition to an agent, preferably a bioactive agent, and a phospholipid (e.g., in one preferred embodiment, an asymmetric phospholipid), the particles can further include a variety of materials. Both inorganic and organic materials can be used. Suitable materials can include, but are not limited to, lipids, fatty acids, inorganic salts, amino acids, polyethylene glycol, trehalose, mannitol, lactose, and maltodextrin. Preferred particle compositions are further described below.

Practice of the present invention provides several advantages. For example, the present invention is directed to particle formulations suitable for inhalation therapy wherein a therapeutic, prophylactic or diagnostic agent is released in a sustained fashion into systemic and/or local circulation.

Additionally, practice of the present invention can provide a method of drug delivery to the pulmonary system wherein the high initial release of agent typically seen in inhalation therapy can be reduced. Consequently, patient compliance and comfort can be increased by not only reducing the frequency of dosing, but also by providing a therapy which is more amenable to patients.

The present invention is directed to the delivery of a bioactive agent via the pulmonary system. In particular, the present invention is directed to particles which comprise a therapeutic, diagnostic or prophylactic agent and an asymmetric phospholipid and which have sustained drug release kinetics and/or therapeutic action. In one embodiment, the particles comprise a therapeutic, prophylactic or diagnostic agent and a phospholipid or combination of phospholipids wherein at least one phospholipid is asymmetric. In other embodiments, the particles comprise a therapeutic, prophylactic or diagnostic agent and no more than one phospholipid wherein the phospholipid is asymmetric. The present invention is also directed to particles which comprise a therapeutic, prophylactic or diagnostic agent and a glycerol fatty acid ester or a combination of glycerol fatty acid esters and which have sustained drug release kinetics and/or therapeutic action. In one embodiment, the particles are in the form of a dry powder suitable for inhalation.

In a preferred embodiment of the invention, the bioactive agent is albuterol. Other therapeutic, prophylactic or diagnostic agents, also referred to herein as “bioactive agents,” “therapeutic agents,” “agents,” “medicaments” or “drugs,” or combinations thereof, can be employed. Hydrophilic as well as hydrophobic drugs can be used.

Suitable bioactive agents include both locally as well as systemically acting drugs. Examples include but are not limited to synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which can, for instance, bind to complementary DNA to inhibit transcription, and ribozymes. The agents can have a variety of biological activities, such as vasoactive agents, neuroactive agents, hormones, anticoagulants, immunomodulating agents, cytotoxic agents, prophylactic agents, antibiotics, antivirals, antisense, antigens, antineoplastic agents and antibodies. In some instances, the proteins may be antibodies or antigens which otherwise would have to be administered by injection to elicit an appropriate response. Compounds with a wide range of molecular weight can be used, for example, between about 100 and about 500,000 grams or more per mole.

Proteins are defined as consisting of 100 amino acid residues or more; peptides are less than 100 amino acid residues. Unless otherwise stated, the term “protein” refers to both proteins and peptides. Examples include insulin, other hormones and antibodies. Polysaccharides, such as heparin, can also be administered.

The agents useful in the practice of the invention have a variety of biological activities, such as but not limited to vasoactive agents, neuroactive agents, hormones, anticoagulants, immunomodulating agents, cytotoxic agents, prophylactic agents, diagnostic agents, antibiotics, antivirals, antisense, antigens, antineoplastic agents and antibodies.

Bioactive agents for local delivery within the lung, include agents such as those for the treatment of asthma, chronic obstructive pulmonary disease (COPD), emphysema, or cystic fibrosis (CF). For example, genes for the treatment of diseases such as cystic fibrosis can be administered, as can beta agonists, steroids, anticholinergics, and leukotriene modifiers for asthma.

Examples of agents include but are not limited to, somatostatin, testosterone, progesterone, estradiol, nicotine, fentanyl, norethisterone, clonidine, scopolomine, cromolyn sodium, salmeterol, formoterol, estrone sulfate, and epinephrine.

Proteins, include complete proteins, muteins and active fragments thereof, such as insulin, immunoglobulins, antibodies, cytokines (e.g., lymphokines, monokines, chemokines), interleukins, interferons, erythropoietin, somatostatin, nucleases, tumor necrosis factor, colony stimulating factors, enzymes (e.g. superoxide dismutase, tissue plasminogen activator), tumor suppressors, blood proteins, hormones and hormone analogs, vaccines (e.g., tumoral, bacterial and viral antigens), antigens, blood coagulation factors; growth factors; peptides including but not limited to parathyroid hormone related peptide, protein inhibitors, protein antagonists, and protein agonists, calcitonin; nucleic acids include, for example, antisense molecules, oligonucleotides, and ribozymes. Polysaccharides, such as heparin, can also be administered. Examples of proteins suitable for compositions and methods disclosed herein include but are not limited to proteins selected from the group consisting of calcitonin, erythropoietin (EPO), factor IX, granulocyte colony stimulating factor (G-CSF), granulocyte macrophage colony stimulating factor (GM-CSF), follicle stimulating hormone (FSH), growth hormone, in particular human growth hormone, adrenocorticotropic hormone, luteinizing hormone releasing hormone (LHRH), insulin, interferon alpha, interferon beta, interferon gamma, interleukin somatostatin analog, vasopressin analog, amylin, ciliary neurotrophic factor, growth hormone releasing factor (GRF), insulin-like growth factor, insulinotropin, interleukin-1 receptor antagonist, interleukin-3, interleukin-4, interleukin-6, macrophage colony stimulating factor (M-CSF), nerve growth factor, parathyroid hormone, thymosin alpha 1, factor IIb/IIIa inhibitor, alpha-1 antitrypsin, anti-RSV antibody, deoxyribonuclease (DNase), bactericidal/permeability increasing protein (BPI), anti-CMV antibody, interleukin-1 receptor, interleukin-1 receptor antagonist and muteins, analogs, deletion and substitution variants and pharmaceutically acceptable salts of the foregoing.

Nucleic acid sequences include genes, oligonucleotides, including modified oligonucleotides, antisense molecules which can, for instance, bind to complementary DNA to inhibit transcription, and ribozymes.

Agents which can be delivered by the particles and methods of the invention include but are not limited to dopamine precursors, dopamine agonists or any combination thereof for example, levodopa (L-Dopa), ethosuximide, carbidopa, apomorphine, sopinirole, pramipexole, pergoline, bronaocriptine. The L-Dopa or other dopamine precursor or agonist may be any form or derivative that is biologically active in a patient being treated.

Examples of anticonvulsant agents include but are not limited to diazepam, valproic acid, divalproate sodium, phenytoin, phenytoin sodium, cloanazepam, primidone, phenobarbital, phenobarbital sodium, carbamazepine, amobarbital sodium, methsuximide, metharbital, mephobarbital, mephenytoin, phensuximide, paramethadione, ethotoin, phenacemide, secobarbitol sodium, clorazepate dipotassium, trimethadione. Other anticonvulsant agents include, for example, acetazolamide, carbamazepine, chlormethiazole, clonazepam, clorazepate dipotassium, diazepam, dimethadione, estazolam, ethosuximide, flunarizine, lorazepam, magnesium sulfate, medazepam, melatonin, mephenytoin, mephobarbital, meprobamate, nitrazepam, paraldehyde, phenobarbital, phenytoin, primidone, propofol, riluzole, thiopental, tiletamine, trimethadione, valproic acid, vigabatrin. Other examples include, but are not limited to, alprazolam, chlordiazepoxide, clorazepate dipotassium, estazolam, medazepam, midazolam, triazolam, as well as benzodiazepinones, including anthramycin, bromazepam, clonazepam, devazepide, diazepam, flumazenil, flunitrazepam, flurazepam, lorazepam, nitrazepam, oxazepam, pirensepine, prazepam, and temazepam.

Examples of agents suitable for for providing symptomatic relief for migraines and other conditions include ketoprofen and other NSAIDs including but not limited to aminopyrine, amodiaquine, ampyrone, antipyrine, apazone, aspirin, benzydamine, bromelains, bufexamac, BW-755C, clofazimine, clonixin, curcumin, dapsone, diclofenac, diflunisal, dipyrone, epirizole, etodolac, fenoprofen, flufenamic acid, flurbiprofen, glycyrrhizic acid, ibuprofen, indomethacin, ketorolac, ketorolac tromethamine, meclofenamic acid, mefenamic acid, mesalamine, naproxen, niflumic acid, oxyphenbutazone, pentosan sulfuric polyester, phenylbutazone, piroxicam, prenazone, salicylates, sodium salicylate, sulfasalazine, sulindac, suprofen, sumatriptan and tolmetin.

Other agents include triptans, ergotamine tartrate, propanolol hydrochloride, isometheptene mucate, dichloralphenazone, and others for anti-migraine activity.

Agents administered for example in the treatment of ADHD and other related conditions include, among others, methylpenidate, dextroamphetamine, pemoline, imipramine, desipramine, thioridazine and carbamazepine.

Preferred agents for sleep disorders include but are not limited to alprazolam, chlordiazepoxide, clorazepate dipotassium, estazolam, medazepam, midazolam, triazolam, as well as benzodiazepinones, including anthramycin, bromazepam, clonazepam, devazepide, diazepam, flumazenil, flunitrazepam, flurazepam, lorasepam, nitrazepam, oxazepam, pirenzepine, prazepam, temazepam, triazolam, and zolpidem. Other agents are known to those skilled in the art.

Still more agents include analgesics/antipyretics for example, ketoprofen, flurbiprofen, aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate, and others.

Antianxiety or panic disorder agents include but are not limited to lorazepam, buspirone hydrochloride, prazepam, chlordizepoxide hydrochloride, oxazepam, clorazepate dipotassium, diazepam, hydroxyzine pamoate, hydroxyzine hydrochloride, alprazolam, droperidol, halazepam, chlormezanone, and others.

Examples of antipsychotic agents include haloperidol, loxapine succinate, loxapine hydrochloride, thioridazine, thioridazine hydrochloride, thiothixene, fluphenazine hydrochloride, fluphenazine decanoate, fluphenazine enanthate, trifluoperazine hydrochloride, chlorpromazine hydrochloride, perphenazine, lithium citrate, prochlorperazine, and the like.

One example of an antimonic agent is lithium carbonate while examples of Alzheimer agents include tetra amino acridine, donapezel, and others.

Sedatives/hypnotics agents include barbiturates (e.g., pentobarbital, phenobarbital sodium, secobarbital sodium), benzodiazepines (e.g., flurazepam hydrochloride, triazolam, tomazeparm, midazolam hydrochloride), and others.

Hypoglycemic agents include, for example, ondansetron, granisetron, meclizine hydrochloride, nabilone, prochlorperazine, dimenhydrinate, promethazine hydrochloride, thiethylperazine, scopolamine, and others. Antimotion sickness agents include, for example, cinnorizine.

Agents of particular suitability are:

| | |
|---|---|
| Appetite Stimulant | Dronabinol |
| Diabetes | AC2993 |
| Erectile Dysfunction | Sildenafil |
| Lung Cancer/Vitamin deficiency | Vitamin A |
| Ovulation Stimulant | Urofollitropin |
| Pulmonary Hypertension | Epoprostenol |
| Cough | Lidocaine |
| Rheumatoid Arthritis | Etanercept |
| Sexual Dysfunction/Parkinson's | Apomorphine |
| COPD/CF | Tobramycin, Gentamicin |
| COPD | Fometerol/Ipatropium Bromine, |
| | Trospium |
| Tuberculosis | Rifampin/rifampicin |
| Low Dose Steroid | Fluticasone |

Combinations of agents also can be employed. Other agents suitable for the practice of the instant invention are known to those skilled in the art. For example, see the On-line Physician's Desk Reference at http://consumer.pdr.net/drug_info/index.html.

Those therapeutic agents which are charged, such as most of the proteins, including insulin, can be administered as a complex between the charged therapeutic agent and a molecule of opposite charge. Preferably, the molecule of opposite charge is a charged lipid or an oppositely charged protein.

The particles can include any of a variety of diagnostic agents to locally or systemically deliver the agents following administration to a patient. Any biocompatible or pharmacologically acceptable gas can be incorporated into the particles or trapped in the pores of the particles using technology known to those skilled in the art. The term gas refers to any compound which is a gas or capable of forming a gas at the temperature at which imaging is being performed. In one embodiment, retention of gas in the particles is improved by forming a gas-impermeable barrier around the particles. Such barriers are well known to those of skill in the art.

Other imaging agents which may be utilized include commercially available agents used in positron emission tomography (PET), computer assisted tomography (CAT), single photon emission computerized tomography, x-ray, fluoroscopy, and magnetic resonance imaging (MRI).

Examples of suitable materials for use as contrast agents in MRI include the gadolinium chelates currently available, such as diethylene triamine pentacetic acid (DTPA) and gadopentotate dimeglumine, as well as iron, magnesium, manganese, copper, chromium, technecium, europium, and other radioactive imaging agents.

Examples of materials useful for CAT and x-rays include iodine based materials for intravenous administration, such as ionic monomers typified by diatrizoate and iothalamate, non-ionic monomers such as iopamidol, isohexol, and ioversol, non-ionic dimers, such as iotrol and iodixanol, and ionic dimers, for example, ioxagalte.

Diagnostic agents can be detected using standard techniques available in the art and commercially available equipment.

The amount of therapeutic, prophylactic or diagnostic agent(s) present in the particles can range from about 0.1 to about 40 weight percent. Combinations of bioactive agents also can be employed. In one embodiment, the concentration of the therapeutic, prophylactic or diagnostic agent(s) present in the particles is at least about 0.5, 1, 2, 4 or at least about 6 weight percent. In another embodiment, the amount of therapeutic, prophylactic or diagnostic agent(s) present in the particles is about 1 to about 20 weight percent or about 5 to about 15 weight percent, such as about 5 to about 10 weight percent.

The particles of the present invention comprise an asymmetric phospholipid. “Asymmetric phospholipids” are also known to those experienced in the art as “mixed-chain” or “non-identical chain” phospholipids. Asymmetric phospholipids having headgroups such as phosphatidylcholine, phosphatidylethanolamine, phosphatidylglycerol, and phosphatidic acids may be used. Examples of asymmetric phospholipids include 1-acyl, 2-acyl-sn-glycero-3-phosphocholines and 1-acyl, 2-acyl-sn-glycero-3-phosphoalkanolamines.

The 1-acyl,2-acyl-sn-glycero-3-phosphocholine phospholipids can be represented by Formula I:

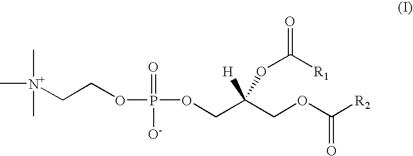

wherein R₁ and R₂ are each independently an aliphatic group having from about 3 to 24 carbon atoms and wherein the aliphatic groups represented by R₁ and R₂ have differing carbon-chain lengths. Preferably, R₁ and R₂ have from about 10 to 20 carbon atoms. An incomplete list of asymmetric phosphatidylcholines and their associated C1 and C2 acyl group carbon-chain lengths appears in Table I.

TABLE I
An Incomplete List of Asymmetric Phosphatidylcholines

| | Common | Chain |
|---|---|---|
| Phospholipid | Name | Lengths |
| 1-Palmitoyl-2-Stearoyl-sn- | PSPC | C16-C18 |
| glycero-3-phosphocholine | | |
| 1-Stearoyl-2-Palmitoyl-sn- | SPPC | C18-C16 |
| glycero-3-phosphocholine | | |
| 1-Stearoyl-2-Myristoyl-sn- | SMPC | C18-C14 |
| glycero-3-phosphocholine | | |
| 1-Myristoyl-2-Stearoyl-sn- | MSPC | C14-C18 |
| glycero-3-phosphocholine | | |
| 1-Myristoyl-2-Palmitoyl-sn- | MPPC | C14-C16 |
| glycero-3-phosphocholine | | |
| 1-Palmitoyl-2-Myristoyl-sn- | PMPC | C16-C14 |
| glycero-3-phosphocholine | | |

“Aliphatic group,” as that term is used herein in reference to Formulas I-V, refers to substituted or unsubstituted straight chained, branched or cyclic C₁-C₂₄ hydrocarbons which can be completely saturated, which can contain one or more heteroatoms such as nitrogen, oxygen or sulfur and/or which can contain one or more units of unsaturation.

Suitable substituents on an aliphatic group include —OH, halogen (—Br, —Cl, —I and —F) —O(aliphatic, substituted), —CN, —NO₂, —COOH, —NH₂, —NH(aliphatic group, substituted aliphatic), —N(aliphatic group, substituted aliphatic group)₂, —COO(aliphatic group, substituted aliphatic group), —CONH₂, —CONH(aliphatic, substituted aliphatic group), —SH, —S(aliphatic, substituted aliphatic group) and —NH—C(═NH)—NH₂. A substituted aliphatic group can also have a benzyl, substituted benzyl, aryl (e.g., phenyl, naphthyl or pyridyl) or substituted aryl group as a substituent. A substituted aliphatic can have one or more substituents.

Specific examples of this type of phospholipid include, but are not limited to, 1-palmitoyl-2-stearoyl-sn-glycero-3-phosphocholine (PSPC); 1-stearoyl-2-palmitoyl-sn-glycero-3-phosphocholine (SPPC); 1-stearoyl-2-myristoyl-sn-glycero-3-phosphocholine (SMPC); 1-myristoyl-2-stearoyl-sn-glycero-3-phosphocholine (MSPC); 1-myristoyl-2-palmitoyl-sn-glycero-3-phosphocholine (MPPC); and 1-palmitoyl-2-myristoyl-sn-glycero-3-phosphocholine (PMPC).

Examples of asymmetric 1-acyl, 2-acyl-sn-glycero-3-phosphoalkanolamine phospholipids include asymmetric 1-acyl, 2-acyl-sn-glycero-3-phosphoethanolamine phospholipids which are represented by Formula II:

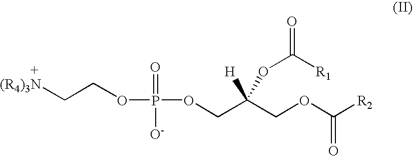

wherein R₁ and R₂ are each independently an aliphatic group having from about 3 to 24 carbon atoms, wherein the aliphatic groups represented by R₁ and R₂ have differing carbon-chain lengths, and R₄ is independently hydrogen or an aliphatic group having from about 1 to 6 carbon atoms. Preferably, R₁ and R₂ have from about 10 to 20 carbon atoms.

Specific examples of this type of phospholipid include, but are not limited to, 1-palmitoyl-2-stearoyl-sn-glycero-3-phosphoethanolamine (PSPE); 1-stearoyl-2-palmitoyl-sn-glycero-3-phosphoethanolamine (SPPE); 1-stearoyl-2-myristoyl-sn-glycero-3-phosphoethanolamine (SMPE); 1-myristoyl-2-stearoyl-sn-glycero-3-phosphoethanolamine (MSPE); 1-myristoyl-2-palmitoyl-sn-glycero-3-phosphoethanolamine (MPPE); and 1-palmitoyl-2-myristoyl-sn-glycero-3-phosphoethanolamine (PMPE).

Particles of the present invention may comprise combinations of asymmetric phospholipids or combinations of asymmetric and symmetric (i.e., identical chain) phospholipids. Alternatively, the particles comprise only one phospholipid (e.g, an asymmetric and a symmetric phospholipid).

In one embodiment of the present invention, particles comprise asymmetric phospholipids having individual acyl chains that are naturally present in the lung. Particles comprising disaturated phospholipids are preferred over particles comprising mono- or di-unsaturated phospholipids.

Without being held to any particular theory, Applicants believe that particles containing asymmetric phospholipids may possess unique packing and/or partition of constituent therapeutic, prophylactic or diagnostic agent molecules, such as albuterol, and result in entrapment or encapsulation of the drug. It is thought that drug release and subsequent uptake of the drug payload from the aerosol formulation will be slower if the drug is entrapped or encapsulated. On the other hand, drug release and subsequent uptake of the drug payload from the aerosol formulation may be faster if the drug is not entrapped or encapsulated, but rather simply surface-associated. Applicants believe that for entrapped or encapsulated drug molecules, the availability of the agent in the dissolution media or physiological lining fluids, such as airway lining fluid (ALF), is not only determined by drug solubility but also by particle dissolution and/or diffusion of drug molecules from the particle matrix. In contrast, it is believed that in particles in which drug molecules are primarily surface associated, the availability of drug molecules is primarily drug solubility limited. Consequently, entrapment or encapsulation of the drug in the particle matrix may slow release and subsequent uptake of the drug.

For identical-chain phosphatidylcholines (PC) in the crystalline state, the equivalent of about 3.68 C—C bond lengths separate the C1 and C2 acyl chain terminals. In a gel-state bilayer described by Huang, et al., the effective carbon-chain length difference is about 1.5 C—C bond lengths. For asymmetric phosphatidylcholines in a gel-state bilayer, the effective chain length difference is about (X−Y+1.5) C—C bond lengths, where X and Y are the C1 and C2 acyl carbon-chain lengths, respectively (Huang, C. and Li, S. “Calorimetric and Molecular Mechanics Studies of the Thermotropic Phase Behavior of Membrane Phospholipids.” Biochim Biophys Acta 1422: 273-307 (1999), the teachings of which are incorporated herein in their entirety). The absolute value of the equation (X−Y+1.5) is expressed as ΔC. The larger the ΔC value, the greater the asymmetry of the phospholipid. Table II lists transition temperature, Tm, and ΔC values for some disaturated asymmetric phosphatidylcholines (PC) and phosphatidylethanolamines (PE). Transition temperatures higher than normal body temperature (about 37° C.) are shown in bold face.

TABLE II
Tm and ΔC values of some disaturated asymmetric
phosphatidylcholines and phosphatidylethanolamines

| | | Carbon-chain | |
|---|---|---|---|
| | Tm of Fully | Length Difference | |
| | Hydrated | between C1 and C2 | ΔC |
| Phospholipid | Samples‡ | Acyl Groups | Value |
| PSPC | 48.8 ° C. | 2 | 0.5 |
| SPPC | 44.4 ° C. | 2 | 3.5 |
| SMPC | 31.2 ° C. | 4 | 2.5 |
| MSPC | 39.2 ° C. | 4 | 6.5 |
| MPPC | 34.9 ° C. | 2 | 0.5 |
| PMPC | 28.4 ° C. | 2 | 3.5 |
| PSPE | 69.6 ° C. | 2 | 0.5 |
| SPPE | 65.9 ° C. | 2 | 3.5 |
| SMPE | 54.9 ° C. | 4 | 2.5 |
| MSPE | 61.6 ° C. | 4 | 6.5 |
| MPPE | 57.7 ° C. | 2 | 0.5 |
| PMPE | 52.3 ° C. | 2 | 3.5 |

‡(Huang, C. and Li, S., “Calorimetric and Molecular Mechanics Studies of the Thermotropic Phase Behavior of Membrane Phospholipids,” Biochim Biophys Acta 1422: 273-307 (1999)).

In their research of phospholipid bilayer membranes, Menger, et al. found that due to the juxtaposition of the C1 acyl terminus of one lipid molecule with the C2 acyl terminus of another lipid molecule from an opposing bilayer leaflet, cavities or pockets may be formed (Menger, F. M. and Wong, Y. -L., “Synthesis of Defective Phospholipids,” J Org Chem, 61:7382-7390 (1996), the teachings of which are incorporated herein in their entirety).

Without being held to any particular theory, Applicants believe that cavities or pockets can form in aerosol particle formulations, that the dimension of the cavities or pockets in aerosol particle formulations can depend on the ΔC value, and that aerosol formulations can be designed for entrapping drug molecules in the asymmetric phospholipid particles. Different phospholipids having different ΔC values may be used to produce aerosol formulations. Applicants believe that the ability of an aerosol formulation to entrap a drug molecule will depend on the size the drug molecule, or, more precisely, the hydrodynamic diameter of the drug molecule. A drug molecule should be small enough to aid efficient entrapment. In a preferred embodiment, particles are formed using an asymmetric phospholipid having a ΔC value of about 0.5 to 9.5. Without being held to any particular theory, Applicants believe that drug molecules are fully entrapped or, alternatively, partially associated within the cavities or pockets contained within the particles of the present invention. Drug molecules that may be entrapped or associated using this approach include, but are not limited to, albuterol sulfate and estrone sulfate. Peptides may also be entrapped or associated in aerosol formulations by altering phospholipid packing and pocket dimension. Particularly suitable aerosol particles are dry powder particles comprising disaturated phospholipids. These particles are preferably aerodynamically light. The process of making aerodynamically light particles, as discussed herein, optimizes the entrapment or association of drug molecules in the cavities or pockets which are hydrophobic.

Particles comprising asymmetric phospholipids are also described in U.S. patent application Ser. No. 60/359,466, entitled “Sustained Release Formulations Utilizing Asymmetric Phospholipids,” filed on Feb. 22, 2002, the contents of which are incorporated herein in their entirety.

The particles and respirable compositions comprising the particles of the invention may comprise a phospholipid or a combination of phospholipids. Examples of suitable phospholipids include, among others, those listed in U.S. patent application Ser. No. 09/665,252 filed on Sep. 19, 2000, described above. Other suitable phospholipids include phosphatidylcholines, phosphatidylethanolamines, phosphatidylglycerols, phosphatidylserines, phosphatidylinositols and combinations thereof. Specific examples of phospholipids include but are not limited to 1,2-dipalmitoyl-sn-glycero phosphocholine (DPPC), 1,2-distearoyl-sn-glycero-3-phosphocholine (DSPC), 1-myristoyl,-2-stearoyl-sn-glycero-3-phosphocholine (MSPC), 1,2-dimyristoyl-sn-glycero-3-phosphoethanolamine (DMPE), 1,2-distearoyl-sn-glycero-3-[phospho-rac-(1-glycerol)] (DSPG), 1,2-dipalmitoyl-sn-glycero-3-phosphoethanolamine (DPPE), or any combination thereof. Other phospholipids are known to those skilled in the art. In a preferred embodiment, the phospholipids are endogenous to the lung.

The particles contain a phospholipid or combination of phospholipids in a concentration of less than about 95, 90, 85 or about 80 weight percent. For example, the phospholipid or combination of phospholipids is present in the particles in an amount ranging from more than about 9 to about 90 weight percent. More commonly, the phospholipid or combination of phospholipids can be present in the particles in an amount ranging from about 40 to about 80 weight percent. In one embodiment, the total phospholipid content is about 60 to about 80 weight percent, such as about 70 to about 80 weight percent, e.g., about 76 weight percent.

In another embodiment of the invention, the phospholipids or combinations thereof are selected to impart controlled release properties to the highly dispersible particles. The phase transition temperature of a specific phospholipid or a combination of phospholipids can be below, around, or above the physiological body temperature of a patient. By selecting phospholipids or combinations of phospholipids according to their phase transition temperature, the particles can be tailored to have controlled release properties. For example, by administering particles which include a phospholipid or combination of phospholipids which have a phase transition temperature higher than the patient's body temperature, the release of the therapeutic, diagnostic or prophylactic agent can be slowed down. On the other hand, rapid release can be obtained by including in the particles phospholipids having lower transition temperatures. Particles having controlled release properties and methods of modulating release of a biologically active agent are described in U.S. Provisional Patent Application No. 60/150,742 entitled “Modulation of Release From Dry Powder Formulations by Controlling Matrix Transition,” filed on Aug. 25, 1999; in U.S. patent application Ser. No. 09/792,869 entitled “Modulation of Release From Dry Powder Formulations,” filed on Feb. 23, 2001; and in International Patent Application No. PCT/US02/05629 entitled “Modulation of Release From Dry Powder Formulations,” filed on Feb. 22, 2002, under Attorney Docket No, 2685.1012-010 and published as WO 02/067902 on Sep. 6, 2002. The contents of these three applications are incorporated by reference in their entirety.

The particles of the present invention can also comprise a charged phospholipid. The term “charged phospholipid,” as used herein, refers to phospholipids which are capable of possessing an overall net charge. The charge on the phospholipid can be negative or positive. The phospholipid can be chosen to have a charge opposite to that of a therapeutic, diagnostic or prophylactic agent when the phospholipid and agent are associated. Preferably, the phospholipid is endogenous to the lung or can be metabolized or processed upon administration to a lung endogenous phospholipid. Combinations of charged phospholipids can be used. The combination of charged phospholipids can also have an overall net charge opposite to that of the therapeutic, diagnostic or prophylactic agent upon association.

In one embodiment, the association of a therapeutic, prophylactic or diagnostic agent and an oppositely charged lipid can result from ionic complexation. In another embodiment, association of a therapeutic, prophylactic or diagnostic agent and an oppositely charged lipid can result from hydrogen bonding. In yet a further embodiment, the association of a therapeutic, prophylactic or diagnostic agent and an oppositely charged lipid can result from a combination of ionic complexation and hydrogen bonding.

The charged phospholipid can be a negatively charged lipid such as, for example, a 1,2-diacyl-sn-glycero-3-[phospho-rac-(1-glycerol)].

The 1,2-diacyl-sn-glycero-3-[phospho-rac-(1-glycerol)] phospholipids can be represented by Formula III:

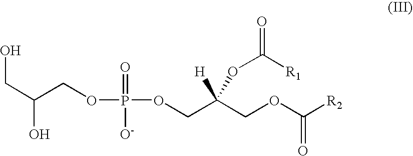

wherein R₁ and R₂ are each independently an aliphatic group having from about 3 to 24 carbon atoms, preferably from about 10 to 20 carbon atoms.

Specific examples of this type of negatively charged phospholipid include, but are not limited to, 1,2-distearoyl-sn-glycero-3-[phospho-rac-(1-glycerol)] (DSPG); 1,2-dimyristoyl-sn-glycero-3-[phospho-rac-(1-glycerol)] (DMPG); 1,2-dipalmitoyl-sn-glycero-3-[phospho-rac-(1-glycerol)] (DPPG); 1,2-dilauroyl-sn-glycero-3-[phospho -rac-(1-glycerol)] (DLPG); and 1,2-dioleoyl-sn-glycero-3-[phospho-rac-(1-glycerol)] (DOPG).

The particles of the invention can also comprise phospholipids which are zwitterionic and therefore do not possess an overall net charge. Such lipids can assist in providing particles with the proper characteristics for inhalation. Such phospholipids suitable for use in the invention include, but are not limited to, 1,2-diacyl-sn-glycero-3-phosphocholines and 1,2-diacyl-sn-glycero-3-phosphoalkanolamines.

The 1,2-diacyl-sn-glycero-3-phosphocholine phospholipids can be represented by Formula IV:

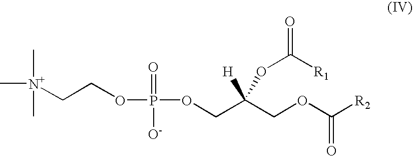

R₁ and R₂ are each independently an aliphatic group having from about 3 to 24 carbon atoms, preferably from about 10 to 20 carbon atoms.

Specific examples of 1,2-diacyl-sn-glycero-3-phosphocholine phospholipids include, but are not limited to, 1,2-dipalmitoyl-sn-glycero-3-phosphocholine (DPPC); 1,2-distearoyl-sn-glycero-3-phosphocholine (DSPC); 1,2-dilaureoyl-sn-3-glycero-phosphocholine (DLPC); 1,2-dimyristoyl-sn-glycero-3-phosphocholine (DMPC); and 1,2-dioleoyl-sn-glycero-3-phosphocholine (DOPC).

Examples of 1,2-diacyl-sn-glycero-3-phosphoalkanolamine phospholipids include 1,2-diacyl-sn-glycero-3-phosphoethanolamine phospholipids which are represented by Formula V:

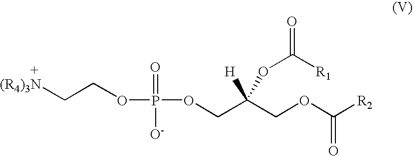

wherein R₁ and R₂ are each independently an aliphatic group having from about 3 to 24 carbon atoms, preferably, from about 10 to 20 carbon atoms and R₄ is independently hydrogen or an aliphatic group having from about 1 to 6 carbon atoms.

Specific examples of this type of phospholipid include, but are not limited to, 1,2-dipalmitoyl-sn-glycero-3-phosphoethanolamine (DPPE); 1,2-dimyristoyl-sn-glycero-3-phosphoethanolamine(DMPE); 1,2-distearoyl-sn-glycero-3-phosphoethanolamine (DSPE); 1,2-dilauroyl-sn-glycero-3-phosphoethanolamine (DLPE); and 1,2-dioleoyl-sn-glycero-3-phosphoethanolamine (DOPE).

In some embodiments, the particles of the present invention comprise components that impart reduced wettability characteristics to the compositions for controlled release pulmonary drug delivery. Without being held to any particular theory, Applicants believe that it is likely that one mechanism of controlled release of a therapeutic, prophylactic or diagnostic agent is based on the reduction of the wettability of the particles'surface. By introducing in the particles'composition a sufficient amount of a hydrophobic material, the release of the drug from the particles can be modulated. The ratio of hydrophobic material(s) concentration to hydrophillic, or wettable, material(s) concentration can be adjusted to produce a desired release rate of the drug from the particles.

Porous particles have relatively high surface areas available for interaction with a release medium such as, for example, alveolar fluid. Particles incorporating appropriate hydrophillic materials such as, for example, glycero-3-l fatty acid esters, can effectively reduce the surface area available for interaction with the release medium and can cause the release of the drug to occur from specific, wettable areas such as those with reduced local concentration of hydrophobic material(s). Furthermore the hydrophobic material, if present in a sufficient concentration, can provide the particles with a more rigid structure that resists degradation and/or dissolution thus providing control of the release of the therapeutic, prophylactic or diagnostic agent by inducing slow erosion of the particle matrix.

Without wishing to be held to any particular theory, Applicants believe that glycerol fatty acid esters impart reduced wettability characteristics to particles for inhalation and thus assist in providing sustained release and/or sustained effect of a therapeutic, prophylactic or diagnostic agent. Hydrophobicity of these compounds is dependent upon both the degree of esterification and the carbon chain length of the compounds. Compounds or mixtures thereof can be provided for use in the instant particles that have varying degrees of esterification and/or carbon chain lengths and thus having varying melting temperatures and/or hydrophilic lipophilic balance (HLB) values. By providing, selecting, or synthesizing compounds having particular melting temperatures and/or hydrophilic lipophilic balance (HLB) values and forming particles comprising said compounds or mixtures thereof, desired wettability characteristics can be imparted to particles for inhalation and thus provide desired or targeted times of release and/or action of a therapeutic, prophylactic or diagnostic agent.

In some embodiments, the particles of the present invention comprise a glycerol fatty acid ester or a combination of fatty acid esters. For example, particles comprise a therapeutic, prophylactic or diagnostic agent, a glycerol fatty acid ester or a combination of fatty acid esters, and a phospholipid or combination of phospholipids. The phospholipid or combination of phospholipids may comprise one or more asymmetric phospholipids.

In one aspect, the particles comprise a glycerol fatty acid ester or combination of glycerol fatty acid esters represented by Structural Formula VI:

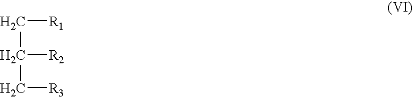

wherein R₁, R₂, and R₃ are, independently, hydroxide or a fatty acid chain and at least one of R₁, R₂, and R₃ is non-hydroxide.

The fatty acid chains can be saturated or unsaturated and branched or unbranched. Examples of saturated, unbranched fatty acid chains for use in the present invention include caprylate, CH₃(CH₂)₆COO⁻; pelargonate, CH₃(CH₂)₇COO⁻; caprate CH₃(CH₂)₈COO⁻; laurate, CH₃(CH₂)₁₀COO⁻; myristate, CH₃(CH₂)₁₃COO⁻; palmitate, CH₃(CH₂)₁₄COO⁻; margarate, CH₃(CH₂)₁₅COO⁻; and stearate CH₃(CH₂)₁₆COO⁻. Examples of unsaturated fatty acids also useful for practice of the invention include palmitoleate, CH₃(CH₂)₅CH═CH(CH₂)₇COO⁻; oleate, CH₃(CH₂)₇CH═CH(CH₂)₇COO⁻; linoleate, CH₃(CH₂)₇CH═CHCH₂CH═CH(CH₂)₇COO⁻; and linolenate, CH₃CH₂CH═CHCH₂CH═CHCH₂CH═CH(CH₂)₇COO⁻.

In another aspect, the particles comprise a glycerol fatty acid ester or combination of glycerol fatty acid esters represented by Structural Formula VII:

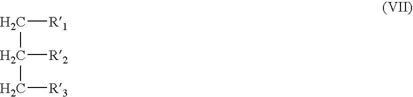

R′₁, R′₂, and R′₃ are, independently, hydroxide, palmitate, or stearate and at least one of R′₁, R′₂, and R′₃ is non-hydroxide. For example, the glycerol fatty acid ester or combination of glycerol fatty acid esters is glyceryl palmitostearate or Precirol® ato 5 (Gattefosse Corporation, Westwood, N.J.), also referred to herein as “Precirol®.” Precirol® ato 5 is synthesized from the esterification of glycerol by palmitostearic acid and is composed of mono-, di- and triglycerides of palmitostearic acid with the diester fraction predominating. Other examples of glycerol fatty acid esters or combinations of glycerol fatty acid esters suitable for use in the present invention include, but are not limited to, tripalmitin, tristearin (e.g. glyceryl stearate available as Precirol® W1 2155 ATO (Gattefosse Corporation, Westwood, N.J.)) and trimyristin.

In one embodiment, the instant particles contain at least about 0.25, 0.5, 1, 3, or at least about 5 weight percent of a glycerol fatty acid ester or combination of glycerol fatty acid esters. For example, the particles contain about 1 to about 60, about 1 to about 40, about 1 to about 30, about 1 to about 25, about 1 to about 15, about 1 to about 10, about 2 to about 8, or about 5 weight percent of a glycerol fatty acid ester or a combination of glycerol fatty acid esters.

Other substances that impart reduced wettability characteristics to the particles of the instant invention include, but are not limited to, substances comprising polyalkylene glycol esters such as, for example, polyethylene glycol esters including, but not limited to, lauroyl macrogloglycerides and stearoyl macrogloglycerides such as Gelucire®. Gelucire® products commonly contain blends of mono-, di-, and or tri-esters of glycerides of long chain fatty acids (e.g., C12 to C18 fatty acids), and polyethylene glycol (PEG) mono- and di-esters of long chain fatty acids (e.g., C12 to C18 fatty acids) and can include free polyethylene glycol (PEG). Gelucire® products are often referred to in the art as polyglycolized glycerides. Examples of Gelucire® include Gelucire® 50/13 and Gelucire® 53/10 (Gattefosse Corporation, Westwood, N.J.). Gelucire® 50/13 and Gelucire® 53/10 are mono-, di-, and tri-glycerides and mono- and di-fatty acid esters of polyethylene glycol 1500. In one embodiment, the instant particles comprise both a glycerol fatty acid ester or a combination of glycerol fatty acid esters and a polyethylene glycol ester or a combination of polyethylene glycol esters. In one example, the particles comprise both Precirol®, such as Precirol® ato 5, and Gelucire®, such as Gelucire® 50/13 or Gelucire® 53/10.

In one embodiment of the invention, particles further comprise one or more amino acids. Hydrophobic amino acids are preferred. Suitable amino acids include naturally occurring and non-naturally occurring hydrophobic amino acids. Some naturally occurring hydrophobic amino acids, including but not limited to, non-naturally occurring amino acids include, for example, beta-amino acids. Both D, L and racemic configurations of hydrophobic amino acids can be employed. Suitable hydrophobic amino acids can also include amino acid analogs. As used herein, an amino acid analog includes the D or L configuration of an amino acid having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. As used herein, aliphatic groups include straight chained, branched or cyclic C1-C8 hydrocarbons which are completely saturated, which contain one or two heteroatoms such as nitrogen, oxygen or sulfur and/or which contain one or more units of desaturation. Aromatic groups include carbocyclic aromatic groups such as phenyl and naphthyl and heterocyclic aromatic groups such as imidazolyl, indolyl, thienyl, furanyl, pyridyl, pyranyl, oxazolyl, benzothienyl, benzofuranyl, quinolinyl, isoquinolinyl and acridintyl.

Suitable substituents on an aliphatic, aromatic or benzyl group include —OH, halogen (—Br, —Cl, —I and —F), —O (aliphatic, substituted aliphatic, benzyl, substituted benzyl, aryl or substituted aryl group), —CN, —NO₂, —COOH, —NH₂, —NH(aliphatic group, substituted aliphatic, benzyl, substituted benzyl, aryl or substituted aryl group), —N(aliphatic group, substituted aliphatic, benzyl, substituted benzyl, aryl or substituted aryl group)₂, —COO(aliphatic group, substituted aliphatic, benzyl, substituted benzyl, aryl or substituted aryl group), —CONH₂, —CONH(aliphatic, substituted aliphatic group, benzyl, substituted benzyl, aryl or substituted aryl group), —SH, —S(aliphatic, substituted aliphatic, benzyl, substituted benzyl, aromatic or substituted aromatic group) and —NH—C(═NH)—NH₂. A substituted benzylic or aromatic group can also have an aliphatic or substituted aliphatic group as a substituent. A substituted aliphatic group can also have a benzyl, substituted benzyl, aryl or substituted aryl group as a substituent. A substituted aliphatic, substituted aromatic or substituted benzyl group can have one or more substituents. Modifying an amino acid substituent can increase, for example, the lypophilicity or hydrophobicity of natural amino acids which are hydrophilic.

A number of the suitable amino acids, amino acids analogs and salts thereof can be obtained commercially. Others can be synthesized by methods known in the art. Synthetic techniques are described, for example, in Green and Wuts, “Protecting Groups in Organic Synthesis,” John Wiley and Sons, Chapters 5 and 7, 1991.

Hydrophobicity is generally defined with respect to the partition of an amino acid between a nonpolar solvent and water. Hydrophobic amino acids are those acids which show a preference for the nonpolar solvent. Relative hydrophobicity of amino acids can be expressed on a hydrophobicity scale on which glycine has the value 0.5. On such a scale, amino acids which have a preference for water have values below 0.5 and those that have a preference for nonpolar solvents have a value above 0.5. As used herein, the term “hydrophobic amino acid” refers to an amino acid that, on the hydrophobicity scale, has a value greater or equal to 0.5, or in other words, has a tendency to partition in the nonpolar acid which is at least equal to that of glycine.

Examples of amino acids which can be employed include, but are not limited to: glycine, proline, alanine, cysteine, methionine, valine, leucine, tyrosine, isoleucine, phenylalanine, tryptophan. Preferred hydrophobic amino acids include leucine, isoleucine, alanine, valine, phenylalanine and glycine. Combinations of hydrophobic amino acids can also be employed. Furthermore, combinations of hydrophobic and hydrophilic (preferentially partitioning in water) amino acids, where the overall combination is hydrophobic, can also be employed.

In one embodiment, the particles of the invention further comprise about 1 to 20 weight percent leucine. In another embodiment, the particles further comprise about 10 to 20 weight percent leucine.

Methods of forming and delivering particles which include an amino acid are described in U.S. patent application Ser. No. 09/382,959, filed on Aug. 25, 1999, entitled “Use of Simple Amino Acids to Form Porous Particles During Spray Drying,” and in U.S. patent application Ser. No. 09/644,320, filed on Aug. 23, 2000, entitled “Use of Simple Amino Acids to Form Porous Particles During Spray Drying,” the teachings of both of which are incorporated herein by reference in their entirety.

In one embodiment, the particles can also include other materials such as, for example, buffer salts, dextran, polysaccharides, lactose, trehalose, mannitol, maltodextrin, cyclodextrins, proteins, peptides, polypeptides, fatty acids, fatty acid esters, inorganic compounds, phosphates, and lipids.

The particles and respirable compositions comprising the particles of the invention may optionally include a surfactant, such as a surfactant which is endogenous to the lung. As used herein, the term “surfactant” refers to any agent which preferentially absorbs to an interface between two immiscible phases, such as the interface between water and an organic polymer solution, a water/air interface or organic solvent/air interface. Surfactants generally possess a hydrophilic moiety and a lipophilic moiety, such that, upon absorbing to microparticles, they tend to present moieties to the external environment that do not attract similarly-coated particles, thus reducing particle agglomeration. Both naturally-occurring and synthetic lung surfactants are encompassed in the scope of the invention.

In addition to lung surfactants, such as, for example, phospholipids discussed above, suitable surfactants include but are not limited to hexadecanol; fatty alcohols such as polyethylene glycol (PEG); polyoxyethylenelauryl ether; surface active fatty acids, such as palmitic acid or oleic acid; glycocholate; surfactin; poloxomers; sorbitan fatty acid esters such as sorbitan trioleate (Span 85); and tyloxapol.

A surfactant can be present in the particles in an amount ranging from more than about 9 to about 90 weight percent. Preferably, a surfactant is present in the particles in an amount of about 50 to 80 weight percent.

In a preferred embodiment, the particles possess aerosol characteristics that permit effective delivery of the particles to the respiratory system without the use of propellents. The terms “respiratory tract” and “respiratory system” are used interchangeably herein.

The particles of the present invention have a preferred size, e.g., a volumetric median geometric diameter (VMGD) of at least about 5 microns. In one embodiment of the invention, the VMGD of the particles is about 5 to about 30 microns. In another embodiment, the particles have a VMGD of about 5 to about 15 microns or, alternatively, about 8 to about 20 microns. In other embodiments, the particles have a median diameter, mass median diameter (MMD), a mass median envelope diameter (MMED) or a mass median geometric diameter (MMGD) of at least about 5 microns, for example from about 5 and about 30 microns.

The diameter of the particles, for example, their VMGD, can be measured using an electrical zone sensing instrument such as a Multisizer IIe, (Coulter Electronic, Luton, Beds, England), or a laser diffraction instrument such as HELOS (Sympatec, Princeton, N.J.). Other instruments for measuring particle geometric diameter are well known in the art. The diameter of particles in a sample will range depending upon factors such as particle composition and methods of synthesis. The distribution of size of particles in a sample can be selected to permit optimal deposition within targeted sites within the respiratory tract.

Particles suitable for use in the present invention may be fabricated or separated, for example, by filtration or centrifugation, to provide a particle sample with a preselected size distribution. For example, greater than about 30, 50, 70, or about 80% of the particles in a sample can have a diameter within a selected range of at least about 5 microns. The selected range within which a certain percentage of the particles must fall may be, for example, between about 5 and about 30 microns or optionally between about 5 and about 15 microns. Optionally, the particle sample also can be fabricated wherein at least about 90% or optionally about 95 or about 99% of the particles, have a diameter within the selected range.

In one embodiment, the interquartile range of the particle sample may be 2 microns with a mean diameter, for example, between about 7.5 and about 13.5 microns. Thus, for example, at least about 30 to about 40% of the particles may have diameters within the selected range. Preferably, the said percentages of particles have diameters within a 1 micron range, for example, between 6 and 7; 10 and 11; 13 and 14; or 14 and 15 microns.

Particle aerodynamic diameter can also be used to characterize the aerosol characteristics of a composition. In one embodiment, the particles have a mass median aerodynamic diameter (MMAD) of about 1 to about 5 microns. In another embodiment, the particles have a MMAD of about 1 to about 3 microns. In yet another embodiment, the particles have a MMAD of about 3 to about 5 microns.

Experimentally, aerodynamic diameter can be determined using time of flight (TOF) measurements. For example, an instrument such as the Model 3225 Aerosizer DSP Particle Size Analyzer (Amherst Process Instrument, Inc., Amherst, Mass.) can be used to measure aerodynamic diameter. The Aerosizer measures the time taken for individual particles to pass between two fixed laser beams. The instrument subsequently uses this TOF data to solve a force balance on the particles and aerodynamic diameter is determined based on the relationship

dₐₑᵣ=d√ρ

where dₐₑᵣ is the aerodynamic diameter of the particle; d is the diameter of the particle; and ρis the particle density.

Aerodynamic diameter also can be experimentally determined by employing a gravitational settling method, whereby the time for an ensemble of particles to settle a certain distance is used to infer directly the aerodynamic diameter of the particles. Indirect methods for measuring the mass median aerodynamic diameter are the Andersen Cascade Impactor and the multi-stage liquid impinger (MSLI). The methods and instruments for measuring particle aerodynamic diameter are well known in the art.

In a preferred embodiment of the invention, particles administered to a subject's respiratory tract have a tap density of less than about 0.4 g/cm³. Particles having a tap density of less than about 0.4 g/cm³ are referred to herein as “aerodynamically light.” In another embodiment, the particles have a tap density less than or equal to about 0.3 g/cm³ or less than or equal to about 0.2 g/cm³. In yet another embodiment, the particles have a tap density less than or equal to about 0.1 g/cm³, or less than or equal to about 0.05 g/cm³. Tap density is a measure of the envelope mass density characterizing a particle. The envelope mass density of a particle of a statistically isotropic shape is defined as the mass of the particle divided by the minimum sphere envelope volume within which it can be enclosed. Features which can contribute to low tap density include irregular surface texture and porous structure.

Tap density can be measured by using instruments known to those skilled in the art such as the Dual Platform Microprocessor Controlled Tap Density Tester (Vankel, N.C.) or a GeoPyc™ instrument (Micrometrics Instrument Corp., Norcross, Ga.). Tap density can be determined using the method of USP Bulk Density and Tapped Density, United States Pharmacopia convention, Rockville, Md., 10^{th} Supplement, 4950-4951, 1999.

Aerodynamically light particles have a preferred size, e.g., a volume median geometric diameter (VMGD) of at least about 5 microns. In one embodiment of the invention, the VMGD of the particles is from about 5 to about 30 microns. Aerodynamically light particles also preferably have a mass median aerodynamic diameter (MMAD), also referred to herein as “aerodynamic diameter,” between about 1 and about 5 microns. In one embodiment of the invention, the MMAD of the particles is between about 1 and about 5 microns.

Process conditions as well as inhaler efficiency, in particular with respect to dispersibility, can contribute to the size of particles that can be delivered to the pulmonary system. Aerodynamically light particles may be fabricated or separated, for example by filtration or centrifugation, to provide a particle sample with a preselected size distribution.

Aerodynamically light particles with a tap density less than about 0.4 g/cm³, median diameters of at least about 5 microns, and an aerodynamic diameter of between about 1 and about 5 microns, preferably between about 1 and about 3 microns, are more capable of escaping inertial and gravitational deposition in the oropharyngeal region, and are targeted to the airways or the deep lung. The use of larger, more porous particles is advantageous since they are able to aerosolize more efficiently than smaller, denser aerosol particles such as those currently used for inhalation therapies.

In comparison to smaller, relatively dense particles, the larger aerodynamically light particles, preferably having a median diameter of at least about 5 microns, also can potentially more successfully avoid phagocytic engulfment by alveolar macrophages and clearance from the lungs, due to size exclusion of the particles from the phagocytes' cytosolic space. Phagocytosis of particles by alveolar macrophages diminishes precipitously as particle diameter increases beyond about 3 microns. Kawaguchi, H., et al., Biomaterials 7: 61-66 (1986); Krenis, L. J. and Strauss, B., Proc. Soc. Exp. Med., 107: 748-750 (1961); and Rudt, S. and Muller, R. H., J. Contr. Rel., 22: 263-272 (1992). For particles of statistically isotropic shape, such as spheres with rough surfaces, the particle envelope volume is approximately equivalent to the volume of cytosolic space required within a macrophage for complete particle phagocytosis.

Aerodynamically light particles thus are capable of a longer term release of an entrapped agent in the lungs. Following inhalation, aerodynamically light biodegradable particles can deposit in the lungs, and subsequently undergo sustained degradation and drug release, without the majority of the particles being phagocytosed by alveolar macrophages. The drug can be delivered relatively slowly into the alveolar fluid, and at a controlled rate into the blood stream, minimizing possible toxic responses of exposed cells to an excessively high concentration of the drug. The aerodynamically light particles thus are highly suitable for inhalation therapies, particularly in controlled release applications.

The particles may be fabricated with the appropriate material, surface roughness, diameter and tap density for localized delivery to selected regions of the respiratory tract such as the deep lung or upper or central airways. For example, higher density or larger particles may be used for upper airway delivery, or a mixture of varying sized particles in a sample, provided with the same or different therapeutic agent may be administered to target different regions of the lung in one administration. Particles having an aerodynamic diameter ranging from about 3 to about 5 microns are preferred for delivery to the central and upper airways. Particles having an aerodynamic diameter ranging from about 1 to about 3 microns are preferred for delivery to the deep lung.

Inertial impaction and gravitational settling of aerosols are predominant deposition mechanisms in the airways and acini of the lungs during normal breathing conditions. Edwards, D. A., J. Aerosol Sci., 26: 293-317 (1995). The importance of both deposition mechanisms increases in proportion to the mass of aerosols and not to particle (or envelope) volume. Since the site of aerosol deposition in the lungs is determined by the mass of the aerosol (at least for particles of mean aerodynamic diameter greater than approximately 1 micron), diminishing the tap density by increasing particle surface irregularities and particle porosity permits the delivery of larger particle envelope volumes into the lungs, all other physical parameters being equal.

The low tap density particles have a small aerodynamic diameter in comparison to the actual envelope sphere diameter. The aerodynamic diameter, dₐₑᵣ, is related to the envelope sphere diameter, d (Gonda, I., “Physico-chemical Principles in Aerosol Delivery,” in Topics in Pharmaceutical Sciences 1991 (eds. D. J. A. Crommelin and K. K. Midha), pp. 95-117, Stuttgart: Medpharm Scientific Publishers, 1992)), by the formula:

dₐₑᵣ=d√ρ

where the envelope mass ρ is in units of g/cm³. Maximal deposition of monodispersed aerosol particles in the alveolar region of the human lung (˜60%) occurs for an aerodynamic diameter of approximately dₐₑᵣ=3 microns. Heyder, J. et al., J. Aerosol Sci., 17: 811-825 (1986). Due to their small envelope mass density, the actual diameter d of aerodynamically light particles comprising a monodisperse inhaled powder that will exhibit maximum deep-lung deposition is:

d=3/√ρ microns (where ρ<1 g/cm³);

where d is always greater than 3 microns. For example, aerodynamically light particles that display an envelope mass density, ρ=0.1 g/cm³, will exhibit a maximum deposition for particles having envelope diameters as large as 9.5 microns. The increased particle size diminishes interparticle adhesion forces. Visser, J., Powder Technology, 58: 1-10. Thus, large particle size increases efficiency of aerosolization to the deep lung for particles of low envelope mass density, in addition to contributing to lower phagocytic losses.

The aerodynamic diameter is calculated to provide for maximum deposition within the lungs, previously achieved by the use of very small particles of less than about 5 microns in diameter, preferably between about 1 and about 3 microns, which are then subject to phagocytosis. Selection of particles which have a larger diameter, but which are sufficiently light (hence the characterization “aerodynamically light”), results in an equivalent delivery to the lungs, but the larger size particles are not phagocytosed. Improved delivery can be obtained by using particles with a rough or uneven surface relative to those with a smooth surface.

Mass density and the relationship between mass density, mean diameter and aerodynamic diameter are discussed in U.S. patent application Ser. No. 08/655,570, filed on May 24, 1996, which is incorporated herein by reference in its entirety.

Methods of preparing and administering particles which are aerodynamically light and include surfactants, and, in particular phospholipids, are disclosed in U.S. Pat. No. 5,855,913, issued on Jan. 5, 1999 to Hanes et al. and in U.S. Pat. No. 5,985,309, issued on Nov. 16, 1999 to Edwards et al. The teachings of both are incorporated herein by reference in their entirety.

Highly dispersible particles suitable for use in the methods of the invention may be prepared using single and double emulsion solvent evaporation, spray drying, solvent extraction, solvent evaporation, phase separation, simple and complex coacervation, interfacial polymerization, supercritical carbon dioxide (CO₂) and other methods well known to those of ordinary skill in the art. Particles may be made using methods for making microspheres or microcapsules known in the art, provided that the conditions are optimized for forming particles with the desired aerodynamic properties (e.g., aerodynamic diameter) or additional steps are performed to select particles with the density and diameter sufficient to provide the particles with an aerodynamic diameter between about 1 and about 5 microns, preferably between about 1 and about 3 microns.

If the particles prepared by any of the methods stated above have a size range outside of the desired range, particles can be sized, for example, using a sieve, and further separated according to density using techniques known to those of skill in the art.

The particles are preferably spray dried. Suitable spray-drying techniques are described, for example, by K. Masters in “Spray Drying Handbook,” John Wiley & Sons, New York, 1984. Generally, during spray-drying, heat from a hot gas such as heated air or nitrogen is used to evaporate a solvent from droplets formed by atomizing a continuous liquid feed.

In a preferred embodiment, a rotary atomizer is employed. An example of a suitable spray dryer using rotary atomization is the Mobile Minor Spray Dryer, manufactured by Niro, Inc. (Denmark). The hot gas can be, for example, air, nitrogen or argon.

In one embodiment, the particles of the invention are obtained by spray drying using an inlet temperature between about 100° C. and about 250° C. and an outlet temperature between about 35° C. and about 100° C. In preferred embodiments, the inlet temperature is about 100° C. to about 120° C. or about 105° C. to about 115° C., for example about 110° C. In another preferred embodiments, the outlet temperature is about 40° C. to about 75° C. or about 40° C. to about 55° C., for example about 43° C. to about 50° C.

An organic solvent or an aqueous-organic solvent can be employed to form a feed for spray drying the particles of the present invention.

Suitable organic solvents that can be employed include but are not limited to alcohols such as, for example, ethanol, methanol, propanol, isopropanol, butanols, and others. Other organic solvents include but are not limited to perfluorocarbons, dichloromethane, chloroform, ether, ethyl acetate, methyl tert-butyl ether and others.

Co-solvents that can be employed include an aqueous solvent and an organic solvent, such as, but not limited to, the organic solvents as described above. Aqueous solvents include water and buffered solutions. In one embodiment, an ethanol and water co-solvent mixture is used. The ethanol solution to water solution ratio can range from about 1:1 to about 9:1 (by volume). In a preferred embodiment, the co-solvent ratio is about 7 parts ethanol solution to 3 parts water solution (by volume).

In one embodiment, the spray dried particles comprise a hydrophobic amino acid such as leucine. Without being held to any particular theory, it is believed that due to their hydrophobicity and low water solubility, hydrophobic amino acids facilitate the formation of a shell during the drying process when an ethanol/water co-solvent mixture is employed. It is also believed that the amino acids may alter the phase behavior of any phospholipids present in such a way as to facilitate the formation of a shell during the drying process.

In one embodiment, the present invention is directed to a method for delivery via the pulmonary system comprising administering an effective amount of particles to the respiratory tract of a person in need of treatment, prophylaxis or diagnosis. The particles of the invention can be used to provide controlled systemic or local delivery of therapeutic, prophylactic or diagnostic agents to the respiratory tract via aerosolization. Administration of the particles to the lung by aerosolization permits deep lung delivery of relatively large diameter therapeutic aerosols, for example, greater than about 5 microns in median diameter. Porous or aerodynamically light particles, having a geometric size (or mean diameter) in the range of about 5 to about 30 microns, and tap density less than about 0.4 g/cm³, such that they possess an aerodynamic diameter of about 1 to about 3 microns, have been shown to display ideal properties for delivery to the deep lung. Larger aerodynamic diameters, ranging, for example, from about 3 to about 5 microns are preferred, however, for delivery to the central and upper airways.

In one embodiment, particles of the present invention are capable of releasing an agent in a sustained fashion. As such, the particles are said to possess sustained release properties. “Sustained release,” as that term is used herein, refers to an increase in the time period over which an agent is released from a particle comprising an asymmetric phospholipid as compared to the time period over which an agent is released from a particle that does not comprise an asymmetric phospholipid. Alternatively, the term “sustained release” is used herein to refer to an increase in the time period over which an agent is released from a particle comprising a glycerol fatty acid ester or a combination of glycerol fatty acid esters as compared to the time period over which an agent is released from a particle that does not comprise a glycerol fatty acid ester or a combination of glycerol fatty acid esters. For example, a sustained release of albuterol from the particles of the present invention can be a release showing in vivo bronchoprotection out to at least about 4 hours post administration, such as about 5 to 6 hours or more. “Sustained release,” as that term is used herein, may also refer to a reduction in the availability, or burst, of agent typically seen soon after administration. For example, “sustained release” can refer to a reduction in the availability of an agent in the first hour following administration, often referred to as the initial burst.

“Sustained release,” as that term is used herein, may also refer to a higher amount of drug retained or remaining in the particles after the initial burst as compared to an appropriate control. “Sustained release” is also known to those experienced in the art as “modified release,” “prolonged release,” or “extended release.” “Sustained release,” as used herein, also encompasses “sustained action” or “sustained effect.” “Sustained action” and “sustained effect,” as those terms are used herein, can refer to an increase in the time period over which an agent performs its therapeutic, prophylactic or diagnostic activity as compared to an appropriate control. “Sustained action” is also known to those experienced in the art as “prolonged action” or “extended action.”

The particles can be fabricated with a rough surface texture to reduce particle agglomeration and improve flowability of the powder. The spray-dried particles have improved aerosolization properties. The spray-dried particles can be fabricated with features which enhance aerosolization via dry powder inhaler devices, and lead to lower deposition in the mouth, throat and inhaler device.

The term “effective amount,” as used herein, refers to the amount of agent needed to achieve the desired therapeutic, prophylactic or diagnostic effect or efficacy. The actual effective amounts of drug can vary according to the specific drug or combination thereof being utilized, the particular composition formulated, the mode of administration, and the age, weight, condition of the patient, and severity of the symptoms or condition being treated. Dosages for a particular patient can be determined by one of ordinary skill in the art using conventional considerations, for example, by means of an appropriate pharmacological protocol.

The particles of the invention can be employed in compositions suitable for drug delivery via the pulmonary system. For example, such compositions can include the particles and a pharmaceutically acceptable carrier for administration to a patient, preferably for administration via inhalation. The particles can be co-delivered with larger carrier particles, not including a therapeutic agent, the latter possessing mass median diameters for example in the range between about 50 microns and about 100 microns. The particles can be administered alone or in any appropriate pharmaceutically acceptable carrier, such as a liquid, for example saline, or a powder, for administration to the respiratory system.

Particles, including an agent or agents, for example albuterol, are administered to the respiratory tract of a patient in need of treatment, prophylaxis or diagnosis. Administration of particles to the respiratory system can be by means such as those known in the art. For example, particles are delivered from an inhalation device. In a preferred embodiment, particles are administered as a dry powder via a dry powder inhaler (DPI). Metered-dose-inhalers (MDI), nebulizers or instillation techniques also can be employed.

The methods of the invention also relate to administering to the respiratory tract of a subject, particles and/or compositions comprising the particles of the invention, which can be enclosed in a receptacle. As described herein, in certain embodiments, the invention is drawn to methods of delivering the particles of the invention, while in other embodiments, the invention is drawn to methods of delivering respirable compositions comprising the particles of the invention. As used herein, the term “receptacle” includes but is not limited to, for example, a capsule, blister, film covered container well, chamber and other suitable means of storing particles, a powder or a respirable composition in an inhalation device known to those skilled in the art.

In a preferred embodiment, the receptacle is used in a dry powder inhaler. Examples of dry powder inhalers that can be employed in the methods of the invention include but are not limited to, the inhalers disclosed is U.S. Pat. Nos. 4,995,385 and 4,069,819, the Spinhaler® (Fisons, Loughborough, U.K.), Rotahaler® (Glaxo-Wellcome, Research Triangle Technology Park, North Carolina), FlowCaps® (Hovione, Loures, Portugal), Inhalator® (Boehringer-Ingelheim, Germany), and the Aerolizer® (Novartis, Switzerland), Diskhaler® (GlaxoSmithKline, RTP, NC), Diskus® (GlaxoSmithKline, RTP, NC), and others known to those skilled in the art. In one embodiment, the inhaler employed is described in U.S. patent application Ser. No. 09/835,302, entitled “Inhalation Device and Method,” filed on Apr. 16, 2001. The entire contents of this application are incorporated by reference herein.

The invention is also drawn to receptacles which are capsules, for example, capsules designated with a particular capsule size, such as size 2. Suitable capsules can be obtained, for example, from Shionogi (Rockville, Md.). The invention is also drawn to receptacles which are blisters. Blisters can be obtained, for example, from Hueck Foils, (Wall, N.J.). Other receptacles and other volumes thereof suitable for use in the present invention are known to those skilled in the art.

The receptacle encloses or stores particles and/or respirable compositions comprising particles. In one embodiment, the particles and/or respirable compositions comprising particles are in the form of a powder. The receptacle is filled with particles and/or compositions comprising particles, as known in the art. For example, vacuum filling or tamping technologies may be used. Generally, filling the receptacle with powder can be carried out by methods known in the art.

In one embodiment of the invention, the receptacle encloses a mass of particles, especially a mass of highly dispersible particles as described herein. The mass of particles comprises a nominal dose of an agent. As used herein, the phrase “nominal dose” means the total mass of an agent which is present in the mass of particles in the receptacle and represents the maximum amount of agent available for administration in a single breath.

Particles and/or respirable compositions comprising particles are stored or enclosed in the receptacles and are administered to the respiratory tract of a subject. As used herein, the terms “administration” or “administering” of particles and/or respirable compositions refer to introducing particles to the respiratory tract of a subject.

As described herein, in one embodiment, the invention is drawn to a respirable composition comprising carrier particles and an agent. In another embodiment, the invention is drawn to a method of delivering a respirable composition comprising carrier particles and an agent. As used herein, the term “carrier particle” refers to particles which may or may not comprise an agent, and aid in delivery of an agent to a subject's respiratory system, for example, by increasing the stability, dispersibility, aerosolization, consistency and/or bulking characteristics of an agent. It is clear that in certain embodiments the particles of the invention are carrier particles which are capable of being delivered to the respiratory tract of a subject.

It is understood that the particles and/or respirable compositions comprising the particles of the invention which can be administered to the respiratory tract of a subject can also optionally include pharmaceutically-acceptable carriers, as are well known in the art. The term “pharmaceutically-acceptable carrier” as used herein, refers to a carrier which can be administered to a patient's respiratory system without any significant adverse toxicological effects. Appropriate pharmaceutically-acceptable carriers, include those typically used for inhalation therapy (e.g., lactose) and include pharmaceutically-acceptable carriers in the form of a liquid (e.g., saline) or a powder (e.g., a particulate powder). In one embodiment, the pharmaceutically-acceptable carrier comprises particles which have a mean diameter ranging from about 50 to about 200 microns, and in particular lactose particles in this range. It is understood that those of skill in the art can readily determine appropriate pharmaceutically-acceptable carriers for use in administering, accompanying and or co-delivering the particles of the invention.

In one embodiment of the invention, the particles and/or respirable compositions comprising particles, are administered in a single, breath-activated step. As used herein, the phrases “breath-activated” and “breath-actuated” are used interchangeably. As used herein, “a single, breath-activated step” means that particles are dispersed and inhaled in one step. For example, in single, breath-activated inhalation devices, the energy of the subject's inhalation both disperses particles and draws them into the oral or nasopharyngeal cavity. Suitable inhalers which are single, breath-actuated inhalers that can be employed in the methods of the invention include but are not limited to simple, dry powder inhalers disclosed in U.S. Pat. Nos. 4,995,385 and 4,069,819, the Spinhaler® (Fisons, Loughborough, U.K.), Rotahaler® (Glaxo-Wellcome, Research Triangle Technology Park, North Carolina), FlowCaps® (Hovione, Loures, Portugal), Inhalator® (Boehringer-Ingelheim, Germany), and the Aerolizer® (Novartis, Switzerland), Diskhaler® (GlaxoSmithKline, RTP, NC), Diskus® (GlaxoSmithKline, RTP, NC) and others, such as known to those skilled in the art. In one embodiment, the inhaler employed is described in U.S. patent application Ser. No. 09/835,302, entitled “Inhalation Device and Method,” filed on Apr. 16, 2001. The entire contents of this application are incorporated by reference herein.

“Single breath” administration can include single, breath-activated administration, but also administration during which the particles, respirable compositions or powders are first dispersed, followed by the inhalation or inspiration of the dispersed particles, respirable compositions or powders. In the latter mode of administration, additional energy than the energy supplied by the subject's inhalation disperses the particles. An example of a single breath inhaler which employs energy other than the energy generated by the patient's inhalation is the device described in U.S. Pat. No. 5,997,848 issued to Patton et al. on Dec. 7, 1999, the entire teachings of which are incorporated herein by reference.

In a preferred embodiment, the receptacle enclosing the particles, respirable compositions comprising particles or powder is emptied in a single, breath-activated step. In another preferred embodiment, the receptacle enclosing the particles is emptied in a single inhalation. As used herein, the term “emptied” means that at least 50% of the particle mass enclosed in the receptacle is emitted from the inhaler during administration of the particles to a subject's respiratory system. This is also called an “emitted dose.”

Delivery to the pulmonary system of particles in a single, breath-actuated step is enhanced by employing particles which are dispersed at relatively low energies, such as, for example, at energies typically supplied by a subject's inhalation. Such energies are referred to herein as “low.” As used herein, “low energy administration” refers to administration wherein the energy applied to disperse and inhale the particles is in the range typically supplied by a subject during inhaling.

In a preferred embodiment of the invention, the particles administered are highly dispersible. As used herein, the phrase “highly dispersible” particles or powders refers to particles or powders which can be dispersed by a RODOS dry powder disperser (or equivalent technique) such that at about 1 Bar, particles of the dry powder emit from the RODOS orifice with geometric diameters, as measured by a HELOS or other laser diffraction system, that are less than about 1.5 times the geometric particle size as measured at 4 Bar. Highly dispersible powders have a low tendency to agglomerate, aggregate or clump together and/or, if agglomerated, aggregated or clumped together, are easily dispersed or de-agglomerated as they emit from an inhaler and are breathed in by the subject. Typically, the highly dispersible particles suitable in the methods of the invention display very low aggregation compared to standard micronized powders which have similar aerodynamic diameters and which are suitable for delivery to the pulmonary system. Properties that enhance dispersibility include, for example, particle charge, surface roughness, surface chemistry and relatively large geometric diameters. In one embodiment, because the attractive forces between particles of a powder varies (for constant powder mass) inversely with the square of the geometric diameter and the shear force seen by a particle increases with the square of the geometric diameter, the ease of dispersibility of a powder is on the order of the inverse of the geometric diameter raised to the fourth power. The increased particle size diminishes interparticle adhesion forces. (Visser, J., Powder Technology, 58:1-10 (1989)). Thus, large particle size, all other things equivalent, increases efficiency of aerosolization to the lungs for particles of low envelope mass density. Increased surface irregularities, and roughness also can enhance particle dispersibility. Surface roughness can be expressed, for example by rugosity.

Particles suitable for use in the methods of the invention can travel through the upper airways (oropharynx and larynx), the lower airways which include the trachea followed by bifurcations into the bronchi and bronchioli and through the terminal bronchioli which in turn divide into respiratory bronchioli leading then to the ultimate respiratory zone, the alveoli or the deep lung. In one embodiment of the invention, most of the mass of particles deposit in the deep lung. In another embodiment of the invention, delivery is primarily to the central airways. In another embodiment, delivery is to the upper airways.

The term “dose” of agent refers to that amount that provides therapeutic, prophylactic or diagnostic effect in an administration regimen. A dose may consist of more than one actuation of an inhaler device. The number of actuations of an inhaler device by a patient are not critical to the invention and may be varied by the physician supervising the administration.

Aerosol dosage, formulations and delivery systems may be selected for a particular therapeutic application, as described, for example, in Gonda, I. “Aerosols for delivery of therapeutic and diagnostic agents to the respiratory tract,” in Critical Reviews in Therapeutic Drug Carrier Systems, 6: 273-313, 1990; and in Moren, “Aerosol dosage forms and formulations,” in: Aerosols in Medicine. Principles, Diagnosis and Therapy, Moren, et al., Eds, Esevier, Amsterdam, 1985.

Other particles, methods for production of particles, and methods of administering particles are described in U.S. patent application Ser. No. 09/878,146, filed on Jun. 8, 2001, entitled “Method and Apparatus for Producing Dry Highly Efficient Delivery Of A Large Therapeutic Mass Aerosol; ” U.S. patent application Ser. No. 09/837,620, filed on Apr. 18, 2001, entitled “Control Of Process Humidity To Produce Large, Porous Particles;” International Patent Application No. PCT/US02/12320 entitled “Control Of Process Humidity To Produce Large, Porous Particles,” filed on Apr. 17, 2002, and published as WO 02/085326 on Oct. 31, 2002; U.S. patent application Ser. No. 10/300,657, filed on Nov. 20, 2002, entitled “Improved Particulate Compositions for Pulmonary Delivery; ” U.S. patent application Ser. No. 10/300,070, filed on Nov. 20, 2002, entitled “Compositions for Sustained Action Product Delivery and Methods of Use Thereof.” Methods and apparatus for producing dry particles are discussed in U.S. patent application Ser. No. 10/101,563, entitled “Method and Apparatus for Producing Dry Particles,” filed on Mar. 20, 2002. The entirety of each of these applications is incorporated herein by reference.

### EXEMPLIFICATION

### Example 1

Several particle formulations, listed in Table III, where prepared by spray drying. Pre-spray drying solutions were prepared by dissolving the phospholipid(s) in ethanol and the leucine and albuterol sulfate in water. Each solution was then separately heated to about 50° C. The ethanol solution was then mixed with the water solution at a ratio of 70/30 (v/v) ethanol/water. The co-solvent mixtures were clear at 50° C. Final total solute concentration of the solution used for spray drying was about 1 g/L. As an example, the SPPC/DSPC/leucine/albuterol sulfate (38/38/16/8) pre-spray drying solution was prepared by dissolving 380 mg SPPC and 380 mg DSPC in 700 mL of ethanol, dissolving 160 mg of leucine and 80 mg of albuterol sulfate in 300 mL of water, heating the solutions separately to 50° C., and then mixing the two solutions to yield one liter of co-solvent with a total solute concentration of 1 g/L (w/v).

Phospholipids were obtained from Avanti Polar Lipids, Inc. (Alabaster, Ala.). Albuterol sulfate and leucine were obtained from Spectrum Quality Products, Inc. (Gardena, Calif.).

The pre-spray drying solution was then used to produce dry powders. A spray dryer with a compressed air driven rotary atomizer operating at 34000 rpm was used. Liquid feed at a rate of 70 mL/min was pumped continuously by a peristaltic pump to the atomizer. Dry nitrogen gas was used as the drying medium. Both the inlet and outlet temperatures were measured. The inlet temperature was controlled manually and was established at 110° C., with a limit of control of about 5° C. The outlet temperature was determined by the inlet temperature and such factors as the gas and liquid feed rates (it varied from about 40° C. to 50° C.). A container was tightly attached to a cyclone for collecting the powder product.

TABLE III
Particle Formulations for Pulmonary Delivery of Albuterol

| Formu- | |
|---|---|
| lation | Composition (% weight basis) |
| A | 76% SPPC; 16% Leucine; 8% Albuterol Sulfate |
| B | 38% SPPC; 38% DSPC; 16% Leucine; 8% Albuterol Sulfate |
| C | 38% SPPC; 38% DPPC; 16% Leucine; 8% Albuterol Sulfate |
| D | 76% MSPC; 16% Leucine; 8% Albuterol Sulfate |
| E | 38% MSPC; 38% DPPC; 16% Leucine; 8% Albuterol Sulfate |
| F | 38% MSPC; 38% DSPC; 16% Leucine; 8% Albuterol Sulfate |
| G | 38% MSPC; 38% SPPC; 16% Leucine; 8% Albuterol Sulfate |

### Example 2

The mass median aerodynamic diameter and the volumetric median geometric diameter of the particles produced in Example 1 were measured.

The mass median aerodynamic diameter (MMAD) of the particles was determined using an Aerosizer/Aerodisperser (Amherst Process Instrument, Amherst, Mass.). Approximately 2 mg of powder formulation was introduced into the Aerodisperser and the aerodynamic size was determined by time of flight measurements.

The volumetric median geometric diameter (VMGD) of the particles was measured using a RODOS dry powder disperser (Sympatec, Princeton, N.J.) in conjunction with a HELOS laser diffractometer (Sympatec). Powder was introduced into the RODOS inlet and aerosolized by shear forces generated by a compressed air stream regulated at 2 bar. The aerosol cloud was subsequently drawn into the measuring zone of the HELOS, where it scattered light from a laser beam and produced a Fraunhofer diffraction pattern used to infer the particle size distribution and determine the median value.

Mass median aerodynamic diameter, volumetric median geometric diameter, and calculated tap density for each of the formulations produced in Example 1 are shown in Table IV below. The powders produced are respirable, as indicated by the physical characteristics of the powders shown in Table IV.

TABLE IV
Physical Properties of Example 1 Particle Formulations

| Formu- | | MMAD | VMGD | Tap Density |
|---|---|---|---|---|
| lation | Composition | (μm) | (μm) | (g/cc) |
| A | SPPC/Leucine/Albuterol | 2.60 | 15.69 | 0.027 |
| | Sulfate | | | |
| B | SPPC/DSPC/Leucine/ | 3.04 | 8.29 | 0.134 |
| | Albuterol Sulfate | | | |
| C | SPPC/DPPC/Leucine/ | 2.96 | 12.35 | 0.057 |
| | Albuterol Sulfate | | | |
| D | MSPC/Leucine/Albuterol | 3.02 | 16.22 | 0.035 |
| | Sulfate | | | |
| E | MSPC/DPPC/Leucine/ | 2.66 | 15.94 | 0.028 |
| | Albuterol Sulfate | | | |
| F | MSPC/DSPC/Leucine/ | 3.16 | 15.54 | 0.041 |
| | Albuterol Sulfate | | | |
| G | MSPC/SPPC/Leucine/ | 2.41 | 15.88 | 0.023 |
| | Albuterol Sulfate | | | |

### Example 3

Particles having compositions as listed in Table III were produced using the method described in Example 1. These particles were then evaluated for bronchoprotection in a guinea pig model of airway hyperresponsiveness. Bronchoprotection provided by the Example 1 powders was compared to that provided by a liquid aerosol albuterol sulfate preparation.

Dry powder formulations and the liquid aerosol control treatments were delivered to anesthetized animals by intratracheal insufflation. Male Hartley guinea pigs were obtained from ElmHill Breeding Laboratories, Inc. (Chemsford, Mass.). The animals were in good health upon arrival and remained so until use; no clinical signs of illness were observed at any time. The temperature in the animal room was ambient room temperature of approximately 70° F. and the ambient humidity was in the range of approximately35-60%. Animals were housed in accordance with the Guide for the Care and Use of Laboratory Animals (ILAR).

Powder formulations were delivered to the pulmonary airways and parenchyma using a Penn-Century (Philadelphia, Pa.) dry powder intratracheal insufflation device. For delivery of liquid aerosols to the same regions, a Penn-Century liquid insufflation device was used. In both cases, a nominal dose of 25 micrograms albuterol sulfate was used.

A BUXCO Unrestrained Whole-Body Plethysmography system was used to assess bronchoprotection (BUXCO Electronics, Inc., Sharon, Conn.). The whole-body plethysmography system measures bronchoconstriction based on the shape of the respiration waveform in the chamber. The enhanced pause value (PenH), a flow-based indicator of airway resistance, was used as an indicator of bronchoprotection. A significant increase in this value indicated significant bronchoconstriction, while prevention of this increase in response to methacholine indicated bronchoprotection. Airway hyperresponsiveness in normal animals to nebulized methacholine was assessed using the BUXCO system both prior to dosing (i.e., as an assessment of baseline airway hyperresponsiveness) and also at discrete time points following administration.

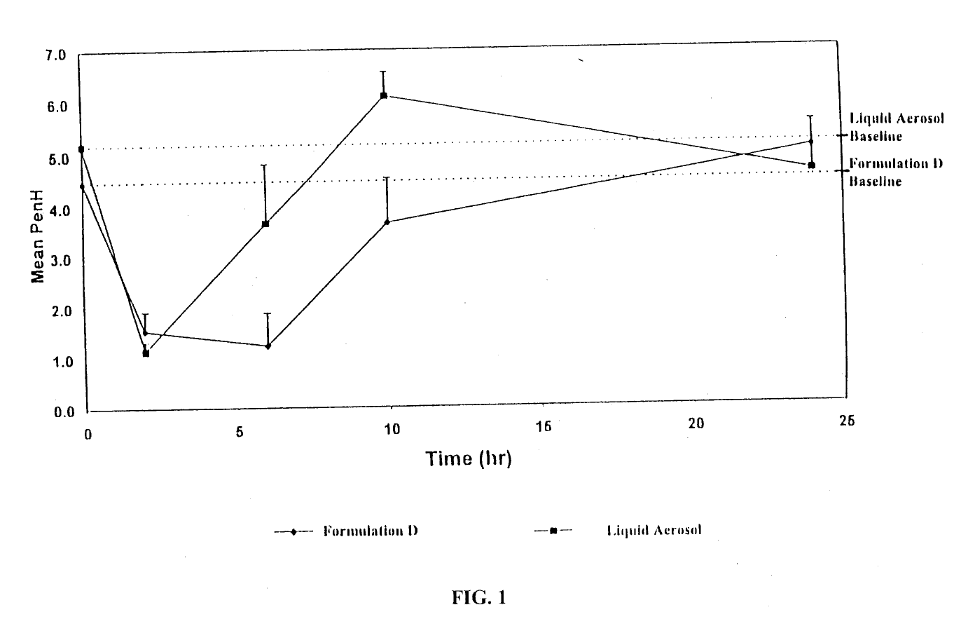

FIG. 1 shows that a nominal dose of 25 micrograms of albuterol sulfate from MSPC-containing Formulation D provided a longer duration of bronchoprotection compared to the same nominal dose of albuterol sulfate from a liquid aerosol preparation at 6 and 10 hours.

### Example 4

Dry powder powders having compositions indicated in Table V were prepared using methods similar to the methods described in Example 1. The mass median aerodynamic diameter (MMAD) and volumetric median geometric diameter (VMGD) of each of the powders were measured as in Example 2 and are shown, along with tap density, in Table V.

TABLE V
Asymmetric phospholipid containing dry particle formulations

| Formu- | | MMAD | VMGD | Density |
|---|---|---|---|---|
| lation | Composition (weight %) | (μm) | (μm) | (g/cc) |
| H | 76% MSPC, 16% leucine, 8% | 2.784 | 12.64 | 0.049 |
| | albuterol sulfate | | | |
| I | 76% MSPC, 16% leucine, 8% | 2.893 | 10.54 | 0.075 |
| | albuterol sulfate | | | |
| J | 76% MSPC, 16% leucine, 8% | 2.767 | 14.41 | 0.037 |
| | albuterol sulfate | | | |
| K | 38% MSPC, 38% DPPC, 16% | 2.924 | 15.85 | 0.034 |
| | leucine, 8% albuterol sulfate | | | |
| L | 38% MSPC, 38% DSPC, 16% | 2.407 | 15.14 | 0.025 |
| | leucine, 8% albuterol sulfate | | | |
| M | 40% MSPC, 60% DPPC | ND | ND | ND |
| N | 12% MSPC, 35% DMPE, 53% | 2.640 | 9.25 | 0.081 |
| | leucine | | | |
| Q | 76% PSPC, 16% leucine, 8% | 2.655 | 17.11 | 0.024 |
| | albuterol sulfate | | | |
| P | 38% PSPC, 38% DPPC, 16% | 2.976 | 16.82 | 0.031 |
| | leucine, 8% albuterol sulfate | | | |
| Q | 38% PSPC, 38% DSPC, 16% | 2.739 | 11.02 | 0.062 |
| | leucine, 8% albuterol sulfate | | | |
| R | 38% PSPC, 38% SPPC, 16% | 2.705 | 15.95 | 0.029 |
| | leucine, 8% albuterol sulfate | | | |

ND: Not Determined

### Example 5

Dry powder powders containing estradiol and having the compositions indicated in Table IV were prepared using methods similar to the methods described in Example 1 except that pre-spray drying solutions were prepared by dissolving the phospholipid and estradiol in ethanol and the leucine in water. The mass median aerodynamic diameter (MMAD) and volumetric median geometric diameter (VMGD) of each of the powders were measured as in Example 2 and are shown, along with tap density, in Table IV.

TABLE VI
Dry particle formulations containing estradiol

| Formu- | | MMAD | VMGD | Density |
|---|---|---|---|---|
| lation | Composition (weight %) | (μm) | (μm) | (g/cc) |
| S | 76% DPPC, 16% leucine, 8% | 3.92 | 12.00 | 0.107 |
| | estradiol | | | |
| T | 76% MSPC, 16% leucine, 8% | 4.01 | 12.60 | 0.101 |
| | estradiol | | | |

### Example 6

Glycerol fatty acid esters can impart desired sustained release properties to particles for inhalation. In order to evaluate hydrophobicity of particle compositions, films of DPPC and Precirol were cast on glass slides from methylene chloride solutions of the 2 components in varying solute ratios. The contact angle of a distilled water droplet deposited on these films was measured as an indicator of hydrophobicity of the film composition. Contact angles of the water drops with the film are shown in Table VII. Two contact angle measurements are indicated for each film.

TABLE VII
Contact angle of water drop with a phospholipid-Precirol film

Film

| DPPC | Precirol ATO 5 | |
|---|---|---|
| (weight %) | (weight %) | Contact Angle |
| 100 | 0 | No droplet formation |
| 90 | 10 | No droplet formation |
| 80 | 20 | No droplet formation |
| 70 | 30 | No droplet formation |
| 60 | 40 | No droplet formation |
| 50 | 50 | No droplet formation |

| | | | |
|---|---|---|---|
| 40 | 60 | 17° | 16° |
| 30 | 70 | 24° | 28° |
| 20 | 80 | 32° | 30° |
| 10 | 90 | 33° | 38° |
| 0 | 100 | 103° | 104° |

These experimental results demonstrate that the presence of glycerol fatty acid esters impart significant hydrophobic properties to the film. The level of hydrophobicity was dependent on the concentration of Precirol in the film with measurable levels being achieved when Precirol exceeded 50% of the total mass.

### Example 7

This example demonstrates the release properties of particles comprising glycerol fatty acid esters (Precirol ATO5), a phospholipid and albuterol sulfate as a drug model. Solutions of 85% ethanol and 15% distilled water (%'s by volume) containing the components indicated in Table VIII were made. The dry powder particles were produced by then spray drying those solutions.

TABLE VIII
Formulations of particles containing Precirol ATO 5

| | DPPC | Precirol ATO 5 | Albuterol Sulfate |
|---|---|---|---|
| Formulation | (weight %) | (weight %) | (weight %) |
| U | 66 | 20 | 4 |
| V | 56 | 40 | 4 |
| W | 36 | 60 | 4 |

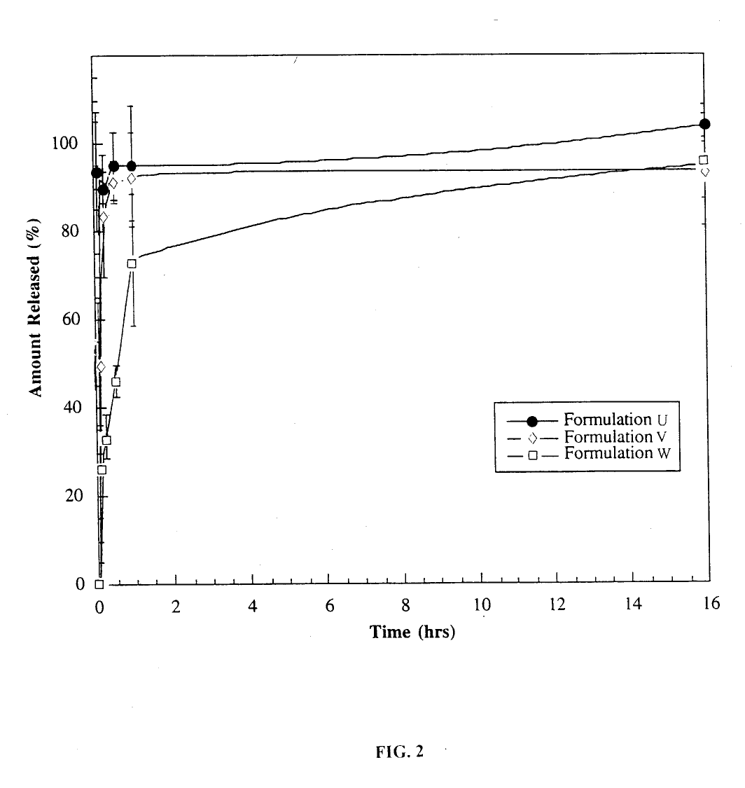

To determine the release rate of albuterol sulfate from the particles, approximately 1-2 mg of the dry powder particles were dispersed in eppendorf tubes containing approximately 1-2 ml of phosphate buffer solution. The concentrations of albuterol sulfate in solution were then measured at timepoints 5 minutes, 15 minutes, 30 minutes 1 hour and 16 hours without stirring and at room temperature. The release profile of albuterol sulfate for the three formulations is shown in FIG. 2.

FIG. 2 demonstrates that the presence of Precirol in Formulation W affects the release profile of albuterol sulfate with only 46% of drug released from the particles in the first 30 minutes of the experiment and only another 27% of the drug releasing in the following 30 minutes. Most of the albuterol sulfate was found in the dissolution medium after 5 minutes for Formulation U and after 15 minutes for Formulation V.

### Example 8

Glycerol fatty acid esters can be used to enhance the sustained release of a therapeutic, prophylactic or diagnostic agent from particles that also comprise one or more asymmetric phospholipids. Several dry powders are made by using the particle production methods of Example 1. Table IX describes the compositions of several of these formulations.

TABLE IX
Dry powder particle formulations for sustained release

| Formulation | Composition (weight %) |
|---|---|
| AA | 56-71% SPPC, 16% leucine, 5-20% Precirol, 8% albuterol |
| | sulfate |
| BB | 28-35.5% SPPC, 28-35.5% DSPC, 16% leucine, 5-20% |
| | Precirol, 8% albuterol sulfate |
| CC | 28-35.5% SPPC, 28-35.5% DPPC, 16% leucine, 5-20% |
| | Precirol, 8% albuterol sulfate |
| DD | 56-71% MSPC, 16% leucine, 5-20% Precirol, 8% albuterol |
| | sulfate |
| EE | 28-35.5% MSPC, 28-35.5% DSPC, 16% leucine, 5-20% |
| | Precirol, 8% albuterol sulfate |
| FF | 28-35.5% MSPC, 28-35.5% DPPC, 16% leucine, 5-20% |
| | Precirol, 8% albuterol sulfate |
| GG | 28-35.5% MSPC, 28-35.5% SPPC, 16% leucine, 5-20% |
| | Precirol, 8% albuterol sulfate |
| HH | 56-71% PSPC, 16% leucine, 5-20% Precirol, 8% albuterol |
| | sulfate |
| II | 28-35.5% PSPC, 28-35.5% DPPC, 16% leucine, 5-20% |
| | Precirol, 8% albuterol sulfate |
| JJ | 28-35.5% PSPC, 28-35.5% DSPC, 16% leucine, 5-20% |
| | Precirol, 8% albuterol sulfate |
| KK | 28-35.5% PSPC, 28-35.5% SPPC, 16% leucine, 5-20% |
| | Precirol, 8% albuterol sulfate |

### Example 9

Several particle formulations comprising a combination phospholipids, leucine, glycerol fatty acid esters (Precirol), and albuterol were produced to evaluate sustained release of the drug in vitro including the effect of an increased glycerol fatty acid ester (Precirol) content. Particles were produced having the compositions shown in Table X. The general method for producing the particles follows. Phospholipids and Precirol were dissolved in an organic phase such as ethanol or isopropyl alcohol (IPA). The alcohol solution containing the phospholipids and Precirol was heated to about 50 to 55° C. to ensure solubilization of these materials and to avoid precipitation of these solutes when the alcohol phase is mixed with an aqueous phase. Leucine and albuterol sulfate were dissolved in an aqueous phase. Aqueous and alcohol phases were mixed on-line using a static mixer or at the atomization nozzle and the resulting solution was atomized and spray dried. Typically, the solvent systems comprised about 20 to 30% water and about 70 to 80% alcohol (%'s by volume). The final solute concentration in the co-solvent system was typically about 1 g/L. The inlet temperature for the spray drier was about 110 to 115° C. and the outlet temperature was about 50 to 55° C. Physical properties of several particle compositions are shown in Table X.

TABLE X
Particle composition and selected physical properties

| | | | | | |
|---|---|---|---|---|---|
| | | | VMGD | VMGD | |
| | | MMAD | 1 bar ¶ | 2 bar ¶ | Density |
| Formulation | Composition (weight %) | (μm) | (μm) | (μm) | (g/cc)‡ |

| | | | | | |
|---|---|---|---|---|---|
| LL | 35.5% DPPC, 35.5% DSPC, 16% | 3.0 | 14.6 | 10.9 | 0.076 |
| | leucine, 5% Precirol, 8% albuterol | | | | |
| | sulfate | | | | |
| MM | 33% DPPC, 33% DSPC, 16% leucine, | 3.2 | 13.2 | 9.6 | 0.111 |
| | 10% Precirol, 8% albuterol sulfate | | | | |
| NN | 33% DPPC, 33% DSPC, 16% leucine, | 3.3 | 9.9 | 7.5 | 0.194 |
| | 10% Precirol, 8% albuterol sulfate | | | | |
| OO | 33% DPPC, 33% DSPC, 16% leucine, | 3.1 | 10.3 | 8.4 | 0.136 |
| | 10% Precirol, 8% albuterol sulfate | | | | |
| PP | 28% DPPC, 28% DSPC, 21% leucine, | 3.0 | 18.4 | 14.1 | 0.045 |
| | 10% Precirol, 8% albuterol sulfate | | | | |
| QQ | 28% DPPC, 28% DSPC, 16% leucine, | 3.2 | 7.0 | 6.2 | 0.266 |
| | 20% Precirol, 8% albuterol sulfate | | | | |

§ Mass median aerodynamic diameter
¶ Parameter median geometric diameter
‡ Based on the equation dₐₑᵣ = d√ρ; using d value at 2 bar pressure

These powders were respirable, as indicated by their physical characteristics shown in Table X. The in vitro performance of the particles was tested for release of the active agent using a Transwell-based release system. Table XI shows the release data for Formulations LL, MM, and QQ.

TABLE XI
In vitro release performance of particle Formulations LL, MM, and QQ

| | Formulation | | |
|---|---|---|---|
| | LL | MM | QQ |
| Time/(min) | (% released) | (% released) | (% released) |
| 5 | 30.5 ± 4. | 35.1 ± 7.3 | 38.3 ± 2.5 |
| 60 | 88.0 ± 5.6 | 101.5 ± 2.4 | 102.7 ± 1.5 |

Following these initial results, a more complete in vitro release test was performed on Formulation LL and the results are reported in Table XII.

TABLE XII
In vitro release performance of particle Formulation LL

| | Amount released |
|---|---|
| Time (min) | (%) |
| 0 | 0 |
| 5 | 34.6 ± 1.6 |
| 10 | 56.6 ± 0.5 |
| 15 | 69.5 ± 2.8 |
| 30 | 84.0 ± 5.2 |
| 60 | 90.3 ± 4.2 |

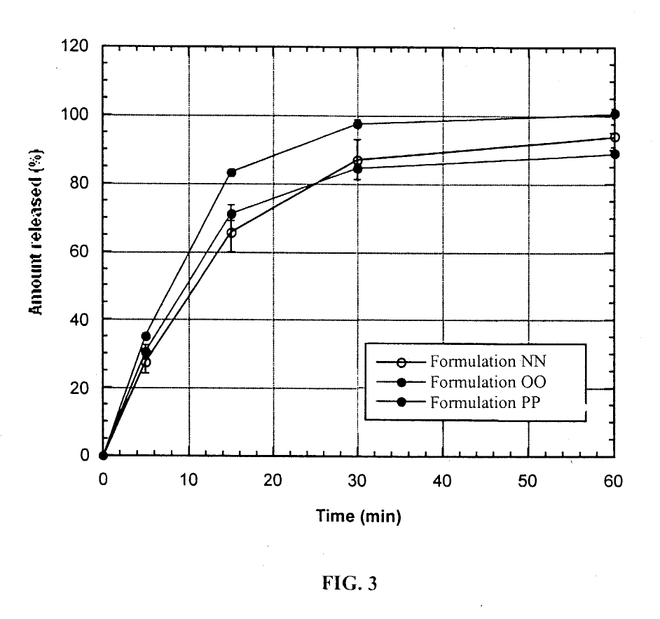

In vitro release performance of Formulations NN, OO, and PP are documented in Table XIII. The data shown in Table XIII are also shown graphically in FIG. 3.

TABLE XIII
In vitro release performance of particle Formulations NN, OO, and PP

| | Formulation | | |
|---|---|---|---|
| | NN | OO | PP |
| Time/(min) | (% released) | (% released) | (% released) |
| 5 | 27.8 ± 3.7 | 30.7 ± 1.8 | 35.0 ± 0.9 |
| 15 | 65.7 ± 5.8 | 71.2 ± 2.2 | 83.2 ± 0.2 |
| 30 | 87.0 ± 5.8 | 84.3 ± 1.2 | 97.4 ± 1.1 |
| 60 | 94.0 ± 2.2 | 89.1 ± 0.1 | 100.6 ± 1.5 |

### Example 10

A non-invasive whole-body plethysmography method for evaluating airway responsiveness in guinea pigs was used, as in Example 3, to test the effectiveness of a powder formulation comprising glycerol fatty acid esters. This animal model allowed repeated assessment of pulmonary function changes in individual guinea pigs challenged at discrete timepoints with nebulized methacholine. A calculated measurement of airway resistance based on flow parameters, PenH (enhanced pause), was used as a marker of protection from methacholine-induced bronchoconstriction. For delivery of dry powder formulations to the pulmonary airways and parenchyma, a Penn-Century dry powder intratracheal insufflation device was used. Dry particles having the composition of Formulation LL (as shown in Table X) were produced using the method of Example 9. Doses of 25 micrograms albuterol sulfate contained in dry powder particles (Formulation LL) were delivered to the pulmonary airways and parenchyma of guinea pigs. For delivery of a liquid aerosol preparation to the same regions, a Penn-Century liquid insufflation device was used.

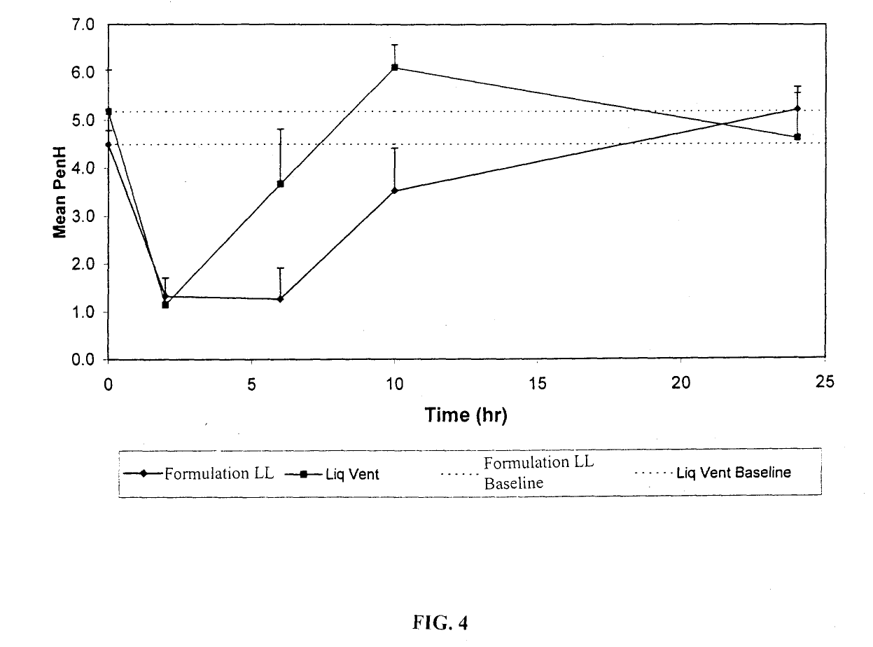

Airway hyperresponsiveness in normal animals to nebulized methacholine was assessed using the BUXCO system both prior to dosing (i.e., as an assessment of baseline airway hyperresponsiveness) and also at discrete time points following administration. As can be seen in FIG. 4, 25 micrograms of albuterol sulfate from Precirol-containing Formulation LL provided a longer duration of bronchoprotection compared to the same dose of albuterol sulfate aerosol from a liquid aerosol preparation (Ventolin® HFA (GlaxoSmithKline, Research Triangle Park, N.C.), also referred to herein as “Liq Vent”) at 6 and 10 hours.

While this invention has been particularly shown and described with references to preferred embodiments thereof, it will be understood by those skilled in the art that various changes in form and details may be made therein without departing from the scope of the invention encompassed by the appended claims.

We claim:

1. Spray dried non-polymeric particles for pulmonary delivery and sustained release of a therapeutic, prophylactic or diagnostic agent comprising:
(a) a therapeutic, prophylactic or diagnostic agent; and
(b) an asymmetric phospholipid;
said particles having a tap density of less than about 0.4 g/cm³.

2. The particles of claim 1 wherein the particles have a tap density less than or equal to about 0.3 g/cm³.

3. The particles of claim 2 wherein the particles have a tap density less than or equal to about 0.2 g/cm³.

4. The particles of claim 3 wherein the particles have a tap density less than or equal to about 0.1 g/cm³.

5. The particles of claim 4 wherein the particles have a tap density less than or equal to about 0.05 g/cm³.

6. The particles of claim 1 wherein the particles have a mean geometric diameter of between about 5 microns and about 30 microns.

7. The particles of claim 6 wherein the particles have a mean geometric diameter of between about 8 microns and 20 microns.

8. The particles of claim 1 wherein the particles have an aerodynamic diameter of between about 1 micron and about 5 microns.

9. The particles of claim 8 wherein the particles have an aerodynamic diameter of between about 1 micron and 3 microns.

10. The particles of claim 8 wherein the particles have an aerodynamic diameter of between about 3 microns and 5 microns.

11. The particles of claim 1 further comprising a compound selected from the group consisting of polysaccharides, sugars, buffer salts, proteins, lipids, surfactants, cholesterol, fatty acids, fatty acid esters and any combination thereof.

12. The particles of claim 1 wherein the particles comprise at least about 2 weight percent of the therapeutic, prophylactic or diagnostic agent.

13. The particles of claim 1 wherein the particles comprise at least about 6 weight percent of the therapeutic, prophylactic or diagnostic agent.

14. The particles of claim 1 wherein the particles comprise about 5 to 10 weight percent of the therapeutic, prophylactic or diagnostic agent.

15. The particles of claim 14 wherein the particles comprise about 8 weight percent of the therapeutic, prophylactic or diagnostic agent.

16. The particles of claim 1 wherein the therapeutic, prophylactic or diagnostic agent is albuterol, or a salt thereof.

17. The particles of claim 1 wherein the therapeutic, prophylactic or diagnostic agent is salmeterol, or a salt thereof.

18. The particles of claim 1 wherein the therapeutic, prophylactic or diagnostic agent is selected from the group consisting of estrone, estradiol, estriol, and salts thereof.

19. The particles of claim 1 wherein the therapeutic, prophylactic or diagnostic agent is a protein or peptide.

20. The particles of claim 1 wherein the therapeutic, prophylactic or diagnostic agent is hydrophilic.

21. The particles of claim 1 wherein the therapeutic, prophylactic or diagnostic agent is hydrophobic.

22. The particles of claim 1 wherein the asymmetric phospholipid is selected from the group consisting of 1-stearoyl-2-palmitoyl-sn-glycero-3-phosphocholine (SPPC) and 1-myristoyl-2-stearoyl-sn-glycero-3-phosphocholine (MSPC).

23. The particles of claim 1 further comprising an identical, or symmetric, chain phospholipid.

24. The particles of claim 23 wherein the identical chain phospholipid is selected from the group consisting of 1,2-dipalmitoyl-sn-glycero-3-phosphocholine (DPPC) and 1,2-distearoyl-sn-glycero-3-phosphocholine (DSPC).

25. The particles of claim 1 wherein the particles comprise a combination of asymmetric phospholipids.

26. The particles of claim 1 wherein the particles comprise about 70 to 80 weight percent phospholipid or combination of phospholipids.

27. The particles of claim 26 wherein the particles comprise about 76 weight percent phospholipid or combination of phospholipids.

28. The particles of claim 1 further comprising an amino acid.

29. The particles of claim 28 wherein the amino acid is hydrophobic.

30. The particles of claim 28 wherein the amino acid is leucine.

31. The particles of claim 30 wherein leucine is present in a concentration of about 10 to 20 weight percent.

32. A method comprising delivering via the pulmonary system of a patient in need of treatment, prophylaxis or diagnosis an effective amount of the particles of claim 1.

33. A method for delivering a sustained release of a therapeutic, prophylactic or diagnostic via the pulmonary system, the method comprising:
administering to the respiratory tract of a patient in need of treatment, prophylaxis or diagnosis an effective amount of spray dried non-polymeric particles comprising:
(a) a therapeutic, prophylactic or diagnostic agent; and
(b) an asymmetric phospholipid;
said particles having a tap density of less than about 0.4 g/cm³.

34. The method of claim 33 wherein the particles have a tap density less than or equal to about 0.3 g/cm³.

35. The method of claim 34 wherein the particles have a tap density less than or equal to about 0.2 g/cm³.

36. The method of claim 35 wherein the particles have a tap density less than or equal to about 0.1 g/cm³.

37. The method of claim 36 wherein the particles have a tap density less than or equal to about 0.05 g/cm³.

38. The method of claim 33 wherein the particles have a mean geometric diameter of between about 5 microns and about 30 microns.

39. The method of claim 38 wherein the particles have a mean geometric diameter of between about 8 microns and 20 microns.

40. The method of claim 33 wherein the particles have an aerodynamic diameter of between about 1 micron and 5 microns.

41. The method of claim 40 wherein the particles have an aerodynamic diameter of between about 1 micron and 3 microns.

42. The method of claim 40 wherein the particles have an aerodynamic diameter of between about 3 microns and 5 microns.

43. The method of claim 33 wherein the particles further comprise a compound selected from the group consisting of polysaccharides, sugars, buffer salts, proteins, lipids, surfactants, cholesterol, fatty acids, fatty acid esters and any combination thereof.

44. The method of claim 33 wherein the particles comprise at least about 2 weight percent of the therapeutic, prophylactic or diagnostic agent.

45. The method of claim 44 wherein the particles comprise at least about 6 weight percent of the therapeutic, prophylactic or diagnostic agent.

46. The method of claim 33 wherein the particles comprise about 5 to 10 weight percent of the therapeutic, prophylactic or diagnostic agent.

47. The method of claim 46 wherein the particles comprise about 8 weight percent of the therapeutic, prophylactic or diagnostic agent.

48. The method of claim 33 wherein the therapeutic, prophylactic or diagnostic agent is albuterol, or a salt thereof.

49. The method of claim 33 wherein the therapeutic, prophylactic or diagnostic agent is salmeterol, or a salt thereof.

50. The method of claim 33 wherein the therapeutic, prophylactic or diagnostic agent is selected from the group consisting of estrone, estradiol, estriol, and salts thereof.

51. The method of claim 33 wherein the therapeutic, prophylactic or diagnostic agent is a protein or peptide.

52. The method of claim 33 wherein the therapeutic, prophylactic or diagnostic agent is hydrophilic.

53. The method of claim 33 wherein the therapeutic, prophylactic or diagnostic agent is hydrophobic.

54. The method of claim 33 wherein the asymmetric phospholipid is selected from the group consisting of 1-stearoyl-2-palmitoyl-sn-glycero-3-phosphocholine (SPPC) and 1-myristoyl-2-stearoyl-sn-glycero-3-phosphocholine (MSPC).

55. The method of claim 33 wherein the particles further comprise an identical, or symmetric, chain phospholipid.

56. The method of claim 55 wherein the identical chain phospholipid is selected from the group consisting of 1,2-dipalmitoyl-sn-glycero-3-phosphocholine (DPPC) and 1,2-distearoyl-sn-glycero-3-phosphocholine (DSPC).

57. The method of claim 33 wherein the particles comprise a combination of asymmetric phospholipids.

58. The method of claim 33 wherein the particles comprise about 70 to 80 weight percent phospholipid or combination of phospholipids.

59. The method of claim 58 wherein the particles comprise about 76 weight percent phospholipid or combination of phospholipids.

60. The method of claim 33 wherein the particles further comprise an amino acid.

61. The method of claim 60 wherein the amino acid is hydrophobic.

62. The method of claim 60 wherein the amino acid is leucine.

63. The method of claim 62 wherein leucine is present in a concentration of about 10 to 20 weight percent.

64. The method of claim 33 wherein delivery is primarily to the deep lung.

65. The method of claim 33 wherein delivery is primarily to the central airways.

66. The method of claim 33 wherein delivery is primarily to the small airways.

67. The method of claim 33 wherein delivery is primarily to the upper airways.

68. The method of claim 33 wherein administration is via a dry powder inhaler.

69. Spray dried non-polymeric particles for pulmonary delivery and sustained release of a therapeutic, prophylactic or diagnostic agent comprising
(a) about 5 to 15 weight percent albuterol sulfate;
(b) about 70 to 80 weight percent of an asymmetric phospholipid or combination of phospholipids wherein at least one phospholipid is asymmetric; and
(c) about 10 to 20 weight percent leucine;
said particles having a tap density of less than about 0.4 g/cm³.

70. The particles of claim 69 wherein the asymmetric phospholipid is selected from the group consisting of 1-stearoyl-2-palmitoyl-sn-glycero-3-phosphocholine (SPPC) and 1-myristoyl-2-stearoyl-sn-glycero-3-phosphocholine (MSPC).

71. A method for delivering a sustained release of a therapeutic, prophylactic or diagnostic via the pulmonary system, the method comprising:
administering to the respiratory tract of a patient in need of treatment, prophylaxis or diagnosis an effective amount of spray dried non-polymeric particles comprising
(a) about 5 to 15 weight percent albuterol sulfate;
(b) about 70 to 80 weight percent of an asymmetric phospholipid or combination of phospholipids wherein at least one phospholipid is asymmetric; and
(c) about 10 to 20 weight percent leucine;
said particles having a tap density of less than about 0.4 g/cm³.

72. The method of claim 71 wherein the asymmetric phospholipid is selected from the group consisting of 1-stearoyl-2-palmitoyl-sn-glycero-3-phosphocholine (SPPC) and 1-myristoyl-2-stearoyl-sn-glycero-3-phosphocholine (MSPC).

73. Spray dried particles for pulmonary delivery and sustained release of a therapeutic, prophylactic or diagnostic agent comprising:
(a) a therapeutic, prophylactic or diagnostic agent;
(b) an amino acid, or a salt thereof; and
(c) an asymmetric phospholipid;
said particles having a tap density of less than about 0.4 g/cm³.

74. A method for delivering a sustained release of a therapeutic, prophylactic or diagnostic via the pulmonary system, the method comprising:
administering to the respiratory tract of a patient in need of treatment, prophylaxis or diagnosis an effective amount of the spray dried particles of claim 73.

75. Particles for pulmonary delivery of a therapeutic, prophylactic or diagnostic agent, the particles comprising:
(a) a therapeutic, prophylactic or diagnostic agent;
(b) a glycerol fatty acid ester or a combination of glycerol fatty acid esters; and
(c) a phospholipid or combination of phospholipids;
said particles having a tap density of less than about 0.4. g/cm³.

76. The particles of claim 75 wherein the glycerol fatty acid ester or combination of glycerol fatty acid esters is represented by the structural formula 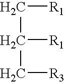
wherein R₁, R₂, and R₃ are, independently, hydroxide, palmitate, or stearate and at least one of R₁, R₂, and R₃ is non-hydroxide.

77. The particles of claim 75 wherein the glycerol fatty acid ester or combination of glycerol fatty acid esters is glyceryl palmitostearate.

78. The particles of claim 75 wherein the glycerol fatty acid ester or combination of glycerol fatty acid esters is present at a concentration of about 1 to about 25 percent by weight.

79. The particles of claim 78 wherein the glycerol fatty acid ester or combination of glycerol fatty acid esters is present at a concentration of about 1 to about 10 percent by weight.

80. The particles of claim 75 further comprising a polyglycolized glyceride.

81. The particles of claim 75 further comprising an amino acid or a salt thereof.

82. The particles of claim 81 wherein the amino acid or salt thereof is leucine.

83. The particles of claim 75 further comprising a material selected from the group consisting of polysaccharides, sugars, polymers, cyclodextrins, lipids, buffer salts, surfactants, cholesterol, fatty acids, fatty acid esters, proteins, peptides, and any combination thereof.

84. The particles of claim 75 wherein the particles are spray dried.

85. The particles of claim 75 wherein the particles have a tap density less than or equal to about 0.3 g/cm³.

86. The particles of claim 85 wherein the particles have a tap density less than or equal to about 0.2 g/cm³.

87. The particles of claim 86 wherein the particles have a tap density less than or equal to about 0.1 g/cm³.

88. The particles of claim 75 wherein the particles have a median geometric diameter of about 5 to about 25 microns.

89. The particles of claim 75 wherein the particles have a median aerodynamic diameter of about 1 to about 5 microns.

90. The particles of claim 89 wherein the particles have a median aerodynamic diameter of about 2 to about 4 microns.

91. A method for delivering a therapeutic, prophylactic or diagnostic to a patient via the pulmonary system, the method comprising:
administering to the respiratory tract of a patient in need of treatment, prophylaxis or diagnosis an effective amount of the particles of claim 75.

92. A method for delivering a sustained release of a therapeutic, prophylactic or diagnostic via the pulmonary system, the method comprising:
administering to the respiratory tract of a patient in need of treatment, prophylaxis or diagnosis an effective amount of particles comprising:
(a) a therapeutic, prophylactic or diagnostic agent;
(b) a glycerol fatty acid ester or a combination of glycerol fatty acid esters; and
(c) a phospholipid or combination of phospholipids;
said particles having a tap density of less than about 0.4. g/cm³.

93. The method of claim 92 wherein the glycerol fatty acid ester or combination of glycerol fatty acid esters is represented by the structural formula 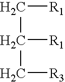
wherein R₁, R₂, and R₃ are, independently, hydroxide, palmitate, or stearate and at least one of R₁, R₂, and R₃ is non-hydroxide.

94. The method of claim 92 wherein the glycerol fatty acid ester or combination of glycerol fatty acid esters is glyceryl palmitostearate.

95. The method of claim 92 wherein the glycerol fatty acid ester or combination of glycerol fatty acid esters is present at a concentration of about 1 to about 25 percent by weight.

96. The method of claim 95 wherein the glycerol fatty acid ester or combination of glycerol fatty acid esters is present at a concentration of about 1 to about 10 percent by weight.

97. The particles of claim 92 further comprising a polyglycolized glyceride.

98. The method of claim 92 wherein the particles further comprise an amino acid or a salt thereof.

99. The method of claim 98 wherein the amino acid or salt thereof is leucine.

100. The method of claim 92 wherein the particles further comprise a material selected from the group consisting of polysaccharides, sugars, polymers, cyclodextrins, lipids, buffer salts, surfactants, cholesterol, fatty acids, fatty acid esters, proteins, peptides, and any combination thereof.

101. The method of claim 92 wherein the particles are spray dried.

102. The method of claim 92 wherein the particles have a tap density less than or equal to about 0.3 g/cm³.

103. The method of claim 102 wherein the particles have a tap density less than or equal to about 0.2 g/cm³.

104. The method of claim 103 wherein the particles have a tap density less than or equal to about 0.1 g/cm³.

105. The method of claim 92 wherein the particles have a median geometric diameter of about 5 to about 25 microns.

106. The method of claim 92 wherein the particles have a median aerodynamic diameter of about 1 to about 5 microns.

107. The method of claim 106 wherein the particles have a median aerodynamic diameter of about 2 to about 4 microns.

108. The method of claim 92 wherein the therapeutic, prophylactic or diagnostic agent has a half time of release from the particles of at least about 15 minutes.

109. The method of claim 92 wherein the therapeutic, prophylactic or diagnostic agent has a half time of release from the particles of at least about 30 minutes.

110. The method of claim 92 wherein particles are delivered primarily to the deep lung.

111. The method of claim 92 wherein particles are delivered primarily to the central airways.

112. The method of claim 92 wherein particles are delivered primarily to the upper airways.

113. The method of claim 92 wherein the particles are administered via a dry powder inhaler.

114. Particles for pulmonary delivery of a therapeutic, prophylactic or diagnostic agent, the particles comprising:
(a) albuterol, or a salt thereof;
(b) glyceryl palmitostearate;
(c) leucine, or a salt thereof;
(d) 1,2-dipalmitoyl-sn-glycero-3-phosphocholine (DPPC); and
(e) 1,2-distearoyl-sn-glycero-3-phosphocholine (DSPC).

115. The particles of claim 114 wherein said particles have a tap density of less than about 0.4 g/cm³.

116. The particles of claim 115 wherein said particles have a tap density of less than about 0.2 g/cm³.

117. The particles of claim 114 wherein the glyceryl palmitostearate is present at a concentration of about 1 to about 10 percent by weight.

118. The particles of claim 114 wherein the albuterol is albuterol sulfate and is present in a concentration of about 5 to about 10 weight percent; the glyceryl palmitostearate is present in a concentration of about 2 to about 8 weight percent; the leucine is present in a concentration of about 13 to about 19 weight percent; and the 1,2-dipalmitoyl-sn-glycero-3-phosphocholine (DPPC) and 1,2-distearoyl-sn-glycero-3-phosphocholine (DSPC) are present in a total concentration of about 65 to about 77 weight percent.

119. A method for delivering a sustained release of a therapeutic, prophylactic or diagnostic via the pulmonary system, the method comprising:
administering to the respiratory tract of a patient in need of treatment, prophylaxis or diagnosis an effective amount of particles comprising:
(a) albuterol, or a salt thereof;
(b) glyceryl palmitostearate;
(c) leucine, or a salt thereof;
(d) 1,2-dipalmitoyl-sn-glycero-3-phosphocholine (DPPC); and
(e) 1,2-distearoyl-sn-glycero-3-phosphocholine (DSPC).

120. The method of claim 119 wherein said particles have a tap density of less than about 0.4 g/cm³.

121. The method of claim 120 wherein said particles have a tap density of less than about 0.2 g/cm³.

122. The method of claim 119 wherein the glyceryl palmitostearate is present at a concentration of about 1 to about 10 percent by weight.

123. The method of claim 119 wherein the albuterol is albuterol sulfate and is present in a concentration of about 5 to about 10 weight percent; the glyceryl palmitostearate is present in a concentration of about 2 to about 8 weight percent; the leucine is present in a concentration of about 13 to about 19 weight percent; and the 1,2-dipalmitoyl-sn-glycero-3-phosphocholine (DPPC) and 1,2-distearoyl-sn-glycero-3-phosphocholine (DSPC) are present in a total concentration of about 65 to about 77 weight percent.
